(19) United States
(12) Patent Application Publication
(10) Pub. No.: US 2009/0029979 A1
(43) Pub. Date: Jan. 29, 2009

(54) 5-HTX MODULATORS

(75) Inventors: Jo Klaveness, Oslo (NO); Bjarne Brudeli, Oslo (NO); Finn Olav Levy, Oslo (NO)

Correspondence Address: Ballard Spahr Andrews & Ingersoll, LLP, SUITE 1000, 999 PEACHTREE STREET, ATLANTA, GA, 30309-3915, US

(73) Assignee: BIO-MEDISINSK INNOVASJON AS, Oslo (NO)

(21) Appl. No.: 11/994,738

(22) PCT Filed: Jul. 7, 2006
(86) PCT No.: PCT/GB2006/002542

§ 371 Date: Sep. 16, 2008

Related U.S. Application Data
(60) Provisional application No. 60/696,780, filed on Jul. 7, 2005.

(51) Int. Cl. A61K 31/5365; C07D 209/04; A61K 31/404; C07D 401/06; A61K 31/517; C07D 401/04; C07D 405/14; A61K 31/496; A61K 31/4439; A61K 31/454; C07D 401/12; C07D 498/04

(52) U.S. Cl. 514/230.2; 548/469; 514/415; 544/285; 514/266.22; 546/277.4; 514/339; 514/323; 546/201; 544/89; 514/253.11; 544/364

(57) ABSTRACT

This invention relates to compounds which bind to serotonin receptors inside or outside the central nervous system, in particular compounds which bind to the 5-HT₂ or 5-HT₇ receptors, their preparation and use, compositions containing them, and methods of treatment using them.

This invention relates to novel compounds which bind to serotonin receptors inside or outside the central nervous system, in particular compounds which bind to the 5-HT₂ or 5-HT₇ receptors, their preparation and use, compositions containing them, and methods of treatment using them.

Serotonin (5-hydroxytryptamine or 5-HT) is a compound with a wide range of activities in the mammalian body. Within the central nervous system it acts as a neurotransmitter, but elsewhere it may act for example as a smooth muscle relaxant or as a vasoconstrictor. The effects of serotinin have thus been linked to a wide range of diseases or malfunctions in the central nervous system, the circulatory system (in particular the heart and blood vessels), the gastrointestinal system, and the bladder.

Serotonin acts through binding with cell-surface receptors (5-HT receptors) and thus its action may be enhanced or counteracted by serotonin agonists or antagonists, compounds which also bind to such receptors.

Until now at least 14 different 5-HT receptors have been identified in mammals which have so far been classified by a IUPAC commission into seven groups (5-HT₁ to 5-HT₇) according to their amino acid sequences and their signal transmission mechanisms. Each receptor group may contain one or more sub-groups, e.g. 5-HT_{1A}, 5-HT_{1B}, 5-HT_{1D}, 5-HT_{1E} and 5-HT_{1F}, and 5-HT receptor modulators, agonists and antagonists alike, have been found to be useful in the treatment of a wide range of conditions such as depression, migraine, nausea, jet lag, cardiac hypertrophy, hypertension, etc.

Since however the same 5-HT receptors may be found both within and outside the central nervous system (CNS), and modulation of such receptors on one side of the blood brain barrier (BBB) may cause a desirable effect while similar modulation (i.e. agonism or antagonism) on the other side may cause an undesirable effect, the present inventors have realised that there is a need for 5-HT receptor modulators which, administered on one side of the BBB do not thereafter cross the BBB there to trigger undesirable effects, e.g. to induce or worsen migraine.

The inventors have thus found that 5-HT receptor modulators which cross the BBB may be produced in analogous forms which do not cross the BBB by derivatization to include oxyacid or oxyacid ester functionalities and may thus be administered in forms which cause the desired effects outside the CNS without simultaneously causing undesired side effects within the CNS. Such compounds are thus particularly suited for use in treatment of disorders of the cardiovascular system, the gastrointestinal system, the musculature, the bladder and of other internal organs other than the brain. Likewise, such compounds if administered directly into the CNS, e.g. by injection or infusion into the cerebrospinal fluid (CSF), may be used in treatment of disorders of the brain without causing undesired peripheral side effects.

Thus, viewed from one aspect the invention provides an oxyacid or oxyacid ester 5-HT receptor modulator or a physiologically tolerable salt thereof, particularly a 5-HT₂ or 5-HT₇ receptor modulator and especially a compound other than a 5-HT₄ receptor modulator, in particular not being a compound disclosed in WO 2005/061483, in particular not a compound as prepared in Examples 3, 4, 7-9, 15-18, 22-24, 26, 27, 29, 31, 32, 40-44, 47, 48, 51-53, 55 or 56 of WO 2005/061483.

The oxyacid ester modulators of the invention may themselves be 5-HT receptor-binders or they may alternatively be bioprecursors for such compounds, e.g. by being transformed by ester cleavage post administration into a 5-HT receptor binding form.

By “oxyacid” is meant herein a group which in its protonated form contains oxygen, hydrogen and an atom selected from C, S and P linked by a double bond to at least one oxygen or, less preferably, sulphur. Thus for example carboxyl (COOH) and its sulphur analogs (CSSH, CSOH and COSH) are covered although carboxyl is preferred. The preferred S oxyacids are SO₃H and OSO₃H, while the preferred P oxyacids are OP(O) (OH)₂ and PO₃H.

Where the compound is in ester form, e.g. containing a group COOR, OP(O) (OR)₂, or SO₂OR, the R group is preferably selected from C₁₋₁₅ alkyl, C₃₋₈ cycloalkyl, aryl, R₂—O—C(O)—R₃—, R₂₀—C(O)—O—R₃—, R₂CO—O—R₃—, R₂CO—O—CO—O—CHR₂—, and R₂—O—CO—O—CO—O—CHR₂— where R₂ and R₃ independently are C₁₋₁₅ alkyl, C₃₋₈ cycloalkyl and aryl groups and the divalent equivalents (i.e. alkylene, etc). In the latter two specified R groups, R₂ is preferably C₁₋₆ alkyl. Unless otherwise specified, alkyl moieties are preferably C₁₋₆ and aryl moieties are single or fused-double homo or heterocyclic rings.

In its protonated form, the oxyacid group preferably has a pka of no more than 6, more preferably no more than 5.5, particularly no more than 5.0, e.g. less than 4.5. In aqueous solution at physiological pH and temperature the oxyacid is preferably at least 90% in deprotonated form, typically at least 95%, especially at least 99%.

Where the oxyacid is in salt form, the counterion may be any physiologically tolerable cation, or where appropriate, anion, e.g. sodium, potassium, calcium, magnesium, ammonium, substituted ammonium, chloride, mesylate, etc. The range of physiologically tolerable counterions is well known in the pharmaceutical literature.

In the compounds of the invention, the oxyacid group may be attached directly or via a linker group to the parent 5-HT receptor molecule. Such linker groups are also conventional in the art and may typically be selected from straight chain or branched optionally substituted C₁₋₁₀ alkyl, C₂₋₁₀ alkenyl and C₂₋₁₀ alkynyl, optionally attached via an amino, oxy, carbonyl, oxycarbonyl, carbonyloxy, aminocarbonyl or carbonylamino group.

In one embodiment, the linker may be interrupted by a heteroatom, preferably sulphur. This heteroatom may be optionally substituted by oxygen such that the interrupting group may be a —S—, —SO— or —SO₂-interrupting group.

In another embodiment, the linker may be interrupted by an optionally substituted aryl group, for example a mono-cyclic aromatic group, preferably a phenyl group.

The oxyacid group in the compounds of the invention is preferably spaced away from the pharmacophore of the parent 5-HT receptor modulator molecule by at least three consecutive bonds, more preferably at least 5.

The point of attachment of the oxyacid group on the parent 5-HT receptor modulator molecules is preferably not at the ring nitrogen of a bicyclic ring system comprising a benzene ring fused to a C₅N unsaturated group as it would then interfere with the pharmacophore. Where the parent group comprises a benzene ring fused to a C₄N₂ ring, the point of attachment may however be directly or indirectly to one of the ring nitrogens, preferably however not one bonded directly to the benzene ring.

The oxyacid group is preferably spaced away from a basic nitrogen of the parent 5-HT receptor modulator by at least three consecutive chemical bonds, preferably at least four such bonds. Where the parent receptor modulator comprises an indole ring or 2-oxa equivalent, the oxyacid group is preferably attached via the 3-position, or if at the 1-position by a group providing at least 6 bonds spacing from the indole ring nitrogen. Where the parent receptor modulator comprises a 4-phenyl-piperazin-1-yl group, the oxyacid group is preferably attached via the 1-position nitrogen. Where the parent receptor modulator comprises an indolinyl or quinazolinyl group, the oxyacid group is preferably attached via the 3-position. Where the parent receptor compound comprises a benzo- or dibenzo-azepinyl group, the oxyacid is preferably attached via the 2-position. Where the parent receptor compound comprises a phthalimide group, the oxyacid group is preferably attached via the phthalimide nitrogen, for example via a group providing at least 5 bonds spacing from the phthalimide nitrogen. Where the parent receptor compound comprises a 1,2,3,4-tetrahydronaphthalene group, or 4-oxo equivalent, the oxyacid group is preferably attached via the 2-position. Where the parent receptor compound comprises an indane group, or 3-oxo equivalent, the oxyacid group is preferably attached via the 1-position. Where the parent receptor compound comprises a 4,5,6,7-tetrahydrobenzofuran group, the oxyacid group is preferably attached via the 5-position. These parent receptor compounds may be used in conjunction with any of the linkers as herein described. Examples of compounds showing the attachment of the oxyacid group to preferred parent receptor compounds are shown in schemes 9 to 11 of Example 82.

In one embodiment the pharmacophore comprises an optionally substituted, multicyclo-alkane, for example a bi- or tri-cycloalkane, preferable adamantane.

The compounds of the invention are preferably oxyacids or oxyacid esters of compounds already proposed for use as 5-HT receptor modulators, in particular compounds which have received regulatory approval in at least one of the USA, Japan and Europe (i.e. EU or an EU member state) or which currently are announced as going through clinical or pre-clinical trials. They may thus be prepared by appropriate modification of the known preparation processes to introduce the oxyacid or oxyacid ester group onto the known framework structure.

Examples of known 5-HT receptor modulators which can be modified in this way to become compounds according to the invention include for example the 5-HT receptor binders discussed by Zefirova et al. in Russian Chemical Reviews 70: 333-355 (2001) and the references therein, the disclosures of which are hereby incorporated by reference. Further examples of 5-HT receptor modulators, especially 5-HT₇, but also 5-HT₅ and 5-HT₆, are discussed by Glennon in J. Medicinal Chemistry 46: 2795-2812 (2003), Wesolowska in Polish J. Pharmacology 54: 327-341 (2002), Vermeulen et al. in J. Medicinal Chemistry 47:5451-5466 (2004), and Thomas et al. in Current Drug Targets—CNS & Neurological Disorder 3: 81-90 (2004), the contents of all of which are hereby incorporated by reference. Particular examples include:
3-(2-aminoethyl)-1H-indole-5-carboxamide;
3-(1,2,3,6-tetrahydropyridin-4-yl)-5-methoxy-1H-indole;
N-[2-[4-(2-methoxyphenyl)-1-piperazinyl]ethyl]-N-2-pyridinyl]cyclohexanecarboxamide;
3-[2-(dimethylamino)ethyl]-N-methyl-1H-indole-5-methanesulfonamide;
3-(2-aminopropyl)-1H-indol-5-ol;
N-[(4-bromophenyl)methyl]-5-methoxy-1H-indole-3-ethanamine;
3-[2-[4-(4-fluorobenzoyl)-1-piperidinyl]ethyl-2,4(1H,3H)-quinazolinedione;
6-(4-methyl-1-piperazinyl)-11H-dibenz[b,e]azepine; and
3-(1-benzyl-1,2,3,6-tetrahydropyridin-4-yl)-5-methoxy-1H-indole.

Typical oxyacid analogs of these specific compounds include:
4N-[3-(2-aminoethyl)-1H-indole-5-carboxamide]butanoic acid;
3-(1-carboxy-1,2,3,6-tetrahydropyridin-4-yl)-5-methoxy-1H-indole;
1-carboxy-4-[N-[2-[4-(2-methoxyphenyl)-1-piperazinyl]ethyl]-N-2-pyridinyl]cyclohexanecarboxamide;
3-[2-(dicarboxymethylamino)ethyl]-N-methyl-1H-indole-5-methanesulfonamide;
4N-[3-(2-aminopropyl)-1H-indol-5-ol]butanoic acid;
N-[(4-carboxyphenyl)methyl]-5-methoxy-1H-indole-3-ethanamine;
3-[2-[4-(4-carboxybenzoyl)-1-piperidinyl]ethyl-2,4(1H, 3H)-quinazolinedione;
6-(4-(4-carboxybutyl)-1-piperazinyl)-11H-dibenz[b,e]azepine; and
3-(1-(4-carboxybenxyl)-1,2,3,6-tetrahydropyridin-4-yl)-5-methoxy-1H-indole.

These compounds are respectively:

5-HT_{1A} agonist; 5-HT_{1A} agonist; 5-HT_{1A} antagonist; 5-HT_{1B} agonist;

5-HT_{2B} agonist; 5-HT_{2B} partial agonist; 5-HT_{2A} antagonist; 5-HT₂ antagonist; and 5-HT₇ partial agonist.

A further aspect of the invention relates to a method of altering the specificity of a 5-HT receptor modulator by the attachment of a suitable moiety or substituent. Suitably modified 5-HT receptor modulators are further described herein and form a further aspect of the invention.

For example, the 5-HT modulatory activity of compounds according to the invention having at least one nucleophilic group on the parent receptor modulator (other than the basic nitrogen required for 5-HT modulatory activity) may be altered or switched between 5-HT receptor subtypes by the attachment of an optionally substituted aromatic moiety, preferably a monocyclic aromatic group optionally substituted by at least one halogen atom, preferably fluorine. p-Fluorobenzene is a particularly preferred moiety. This aspect of the invention is illustrated in scheme 11 of Example 82 in which a 5-HT₇ modulator gains affinity for the 5-HT_{2A} receptor.

The compounds according to the invention preferably fulfil the following requirements: (1) a binding affinity to a 5-HT receptor with a pKᵢ of at least 3, preferably at least 4, more preferably at least 5. Most preferably the compounds have a binding affinity to a 5-HT receptor with a pKᵢ of at least 5; and (2) contain a basic nitrogen atom; and (3) comprise an oxyacid group with a pKₐ of no more than 6.4; or (4) are a compound which is an ester or salt of a compound fulfilling requirements (1), (2) and (3).

It is especially preferred that, insofar as condition (1) is concerned, the binding affinity for 5-HT receptors occurring on the same side of the blood brain barrier but in different 5-HTₙ groups or subgroups has a pKᵢ at least 1.0 smaller, preferably at least 1.5, especially at least 2.0 smaller, i.e. that the compound is specific in its binding to particular 5-HT receptors at the same side of the blood brain barrier. Since the compounds do not cross the BBB to any significant extent, cross-reactivity with 5-HT receptors which only occur on the other side of the BBB, while still not preferred, is acceptable.

Of particular interest are compounds according to the invention which are: 5-HT_{1B} or _{1D} agonists (e.g. to achieve a vasoconstrictive effect outside the CNS); 5-HT_{1B} or _{1D} antagonists (e.g. for use in treatment of hypertension); 5-HT_{2A} antagonists (e.g. for use in treatment of hypertension); 5-HT_{2B} antagonists (e.g. for use in treatment of cardiac hypertrophy, cardiac valve disease or pulmonary hypertension); 5-HT₇ agonists (e.g. for use in treatment of hypertension); and 5-HT₄ receptor modulators for treatment of conditions responsive to 5-HT₄ receptor modulation within the CNS. Of slightly less interest are 5-HT_{1F} receptor modulators, 5-HT_{2C} receptor modulators, 5-HT₃ receptor modulators and 5-HT₆ receptor modulators.

As mentioned above, compounds according to the invention may be prepared by standard chemical modification of the synthesis procedures used for the preparation of the parent receptor modulators, e.g. N-alkylation using a haloalkylcarboxylic acid with a protected carboxyl group followed by carboxyl deprotection.

By way of example the following synthesis scheme may be mentioned.

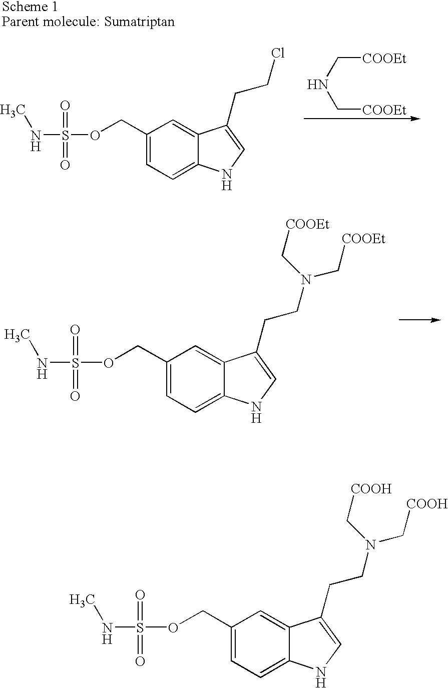

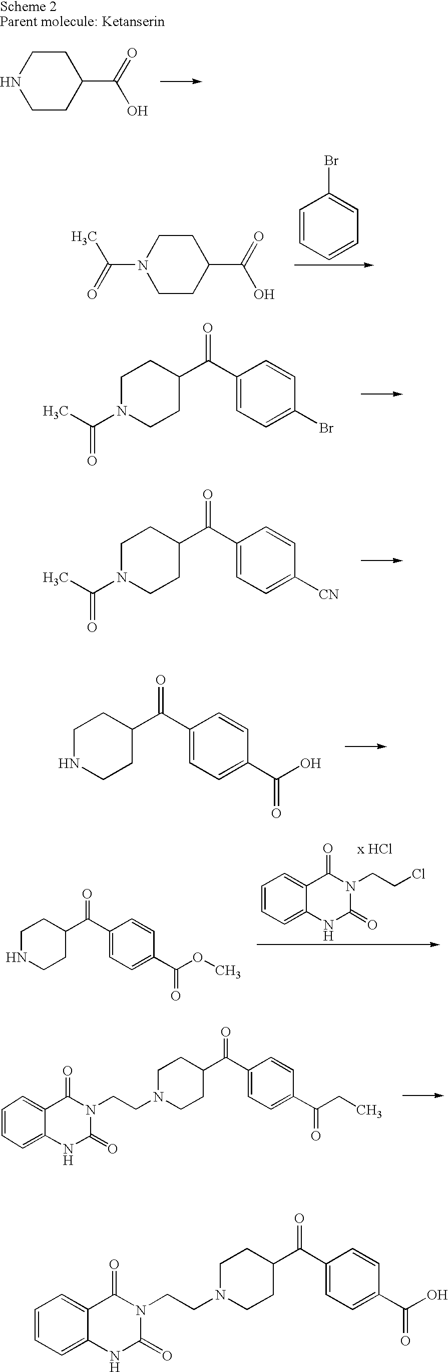

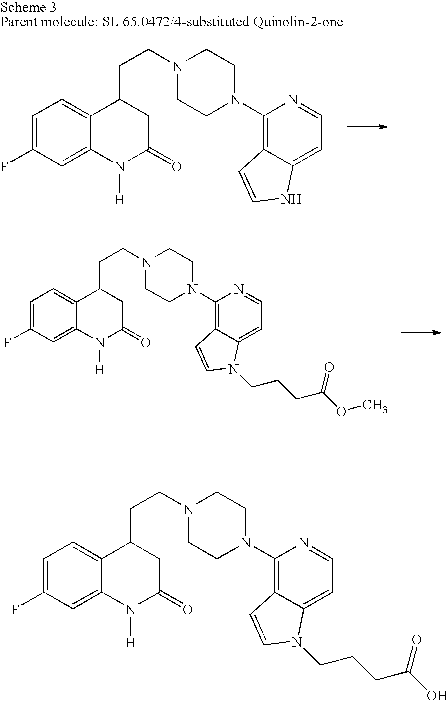

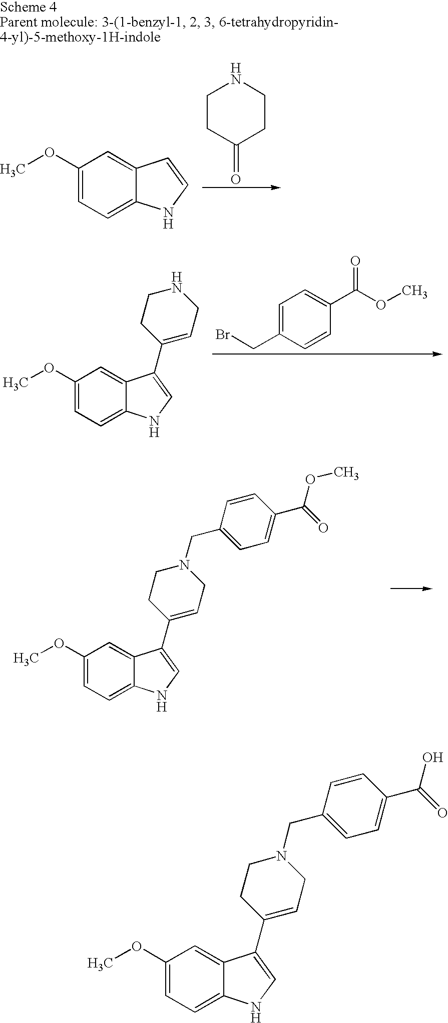

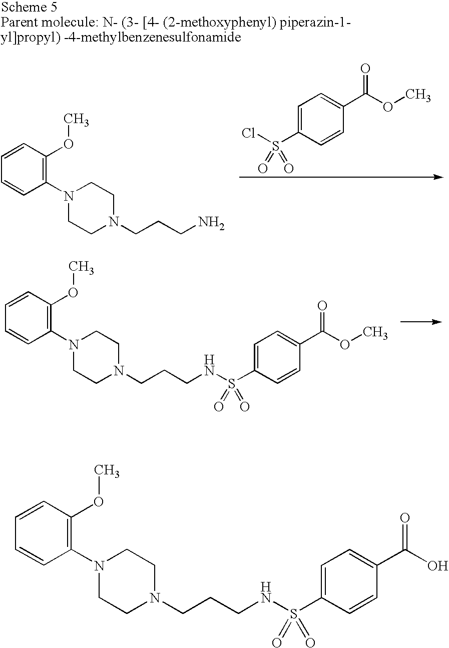

Thus viewed from a further aspect the invention provides a process for the production of a hydrophilic analogue of a 5-HT receptor modulator, e.g. a compound according to the invention, said process comprising (a) reacting said receptor modulator with a bifunctional reagent comprising a modulator binding functional group and an optionally protected oxyacid group; or (b) reacting an intermediate in the preparation of said receptor modulator with a bifunctional reagent comprising an intermediate binding functional group and an optionally protected oxyacid group and optionally further reacting the resultant compound to produce said analogue; and, optionally, (c) removing or replacing the oxyacid protecting groups.

Viewed from a further aspect the invention provides a pharmaceutical composition comprising a receptor modulator or salt thereof according to the invention together with at least one physiologically tolerable carrier or excipient.

The carriers or excipients used in the compositions may be any of the material commonly used in pharmaceutical compositions, e.g. solvents (for example water), pH modifiers, viscosity modifiers, fillers, diluents, binders, aromas, skin penetration enhancers, antioxidants and other preservatives, etc. The choice will depend on the dosage administration route and form. Typically the compositions will be sterile.

The compositions of the invention may be in any convenient dosage administration form, e.g. solutions, dispersions, suspensions, syrups, tablets, coated tablets, powders, sprays, suppositories, etc. Solutions, dispersions and tablets are preferred, especially solutions for injection. These may be prepared in conventional fashion.

The administration route for the compounds and compositions of the invention may be enteral, e.g. oral, rectal or by tube, nasal, sub-lingual, by injection or infusion, e.g. iv, ip, im or s.c. or, less preferably epidural or intracerebroventricular.

Where the compound to be used according to the invention is a 5-HT₄ receptor modulator, it is preferably administered on the CNS side of the BBB, e.g. by intracerebroventricular injection or infusion. This may be done for example to treat conditions relating to the CNS, eg migraine, nausea, depression, etc, without causing unwanted peripheral effects, eg on the cardiovascular or gastrointestinal systems. Suitable 5-HT₄ receptor modulators are disclosed in WO 2005/061483, the contents of which are hereby incorporated by reference.

Viewed from a further aspect the invention provides a receptor modulator or salt thereof according to the invention for medical use.

Viewed from a still further aspect the invention provides the use of a receptor modulator or salt thereof according to the invention for the manufacture of a medicament for use in treating a serotonin-related condition on only one side of the blood brain barrier.

Viewed from a yet still further aspect the invention provides a method of treatment of a human or non-human mammalian subject to contact a serotonin-related condition which method comprises administering on one side of the blood brain barrier, preferably not the central nervous system side, an effective amount of a receptor modulator or salt thereof according to the invention.

The conditions treated according to the method of the invention will generally be conditions responsive to 5-HT receptor agonism or antagonism on one side of the blood brain barrier where corresponding agonism or antagonism of 5-HT receptors on the other side is associated with undesired side effects, e.g. elevated toxicity or undesired CNS effects. Such conditions may be associated for example with disease conditions of the urinary system, the gastrointestinal system, the cardiovascular system, internal organs other than the brain, or (less preferably) the CNS. Examples include hypertension, cardiac hypertrophy, jet lag, nausea and migraine.

Further examples of particular disorders treatable with the compounds of the invention include gastroesophageal reflux, diarrhoea, abdominal cramps, dyspepsia, gastroparesis, constipation, post-operative ileus, intestinal pseudo-obstruction, irritable bowel syndrome, bladder disorders (e.g. hyperactive bladder, etc), hypertension, pulmonary hypertension, portal hypertension, cardiac hypertrophy, cardiac valve disease, etc.

In the method of the invention, the compounds of the invention may typically be administered at dosages of 50 to 200% of the dosages conventional for their parent compounds (i.e. the non-oxyacid or oxyacid ester analogues).

The compounds of the invention have the following particular benefits relative to their parent compounds: lower acute toxicity; improved safety profile; improved toxicity profile relative to effects on the CNS (if administered outside the CNS); and increased efficacy relative to peripheral indications (if administered outside the CNS).

The invention is illustrated further by the following non-limiting Examples:

### EXAMPLE 1

### Preparation of intermediate 1-acetylpiperidin-4-carboxylic acid

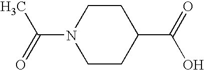

Isonipectoic acid (6.46 g, 50.0 mmol) suspended in CH₂Cl₂ (100 ml) was dropwise added a solution of acetic anhydride (5.61 g, 55.0 mmol) in CH₂Cl₂ (10 ml). The reaction mixture was stirred at room temperature night over and evaporated in vacuo. The residue was recrystallized from EtOH to leave the product as a white solid (7.38 g, 86.2%).

¹H-NMR (300 MHz, DMSO-d₆): δ 12.24 (S, 1H), 4.20-4.16 (m, 1H), 3.75-3.71 (m, 1H), 3.12-3.03 (m. 2H), 2.72-2.64 (m, 1H), 2.50-2.43 (m, 1H), 1.98 (s, 3H), 1.85-1.75 (m, 2H), 1.50-1.45 (m, 1H), 1.45-1.30 (m, 1H)

### EXAMPLE 2

### Preparation of intermediate (4-bromophenyl) (1-acetylpiperidin-4-yl)methanone

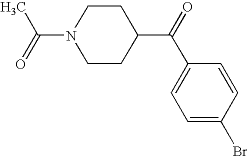

To a stirred solution of SOCl₂ (30 ml) was added 1-acetylpiperidin-4-carboxylic acid (4.38 g, 25.6 mmol) portionwise and stirred at room temperature for 4 hrs. The acid chloride precipitated out of the solution and was filtered off. The solid was washed with Et₂O and dried in vacuum. 1-Acetylpiperidin-4-carboxyl chloride (4.74 g, 25.0 mmol) was slowly added to a solution of AlCl₃ (6.67 g, 50.0 mol) in brombenzene (20 ml). The reaction mixture was heated under reflux for 2 hrs, cooled to room temperature, and poured into crushed ice. The aqueous mixture was extracted with CH₂Cl₂, the organic layer washed with brine and dried over Na₂SO₄, filtered and evaporated in vacuo to leave an oil. The oil was separated with flash chromatography (CH₂Cl₂/MeOH, 9:1) to leave the product as a white solid (4.77 g, 61.6%).

¹H-NMR (300 MHz, CDCl₃): δ 7.76-7.72 (m, 2H), 7.58-7.54 (m, 2H), 4.52-4.47 (m, 1H), 3.86-3.82 (m, 1H), 3.42-3.35 (m, 1H), 3.21-3.12 (m, 1H), 2.80-2.72 (m, 1H), 2.04 (s, 3H), 1.85-1.55 (m, 4H)

### EXAMPLE 3

### Preparation of intermediate (4-cyanophenyl) (1-acetylpiperidin-4-yl)methanone

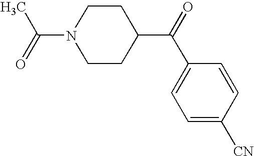

To Zn dust (209 mg, 3.2 mmol) in DMA (23 ml) under argon atmosphere, Br₂ (80:1) was added dropwise and the mixture was stirred at room temperature for ½ h. To the mixture was added Zn(CN)₂ (564 mg, 4.8 mmol), PPh₃ (340 mg, 1.3 mmol), Pd—C₅% (680 mg) and (4-bromophenyl)(1-acetylpiperidin-4-yl)methanone (2.48 g, 8.0 mmol). The mixture was heated at 120° C. overnight, cooled to room temperature and EtOAc (60 ml) was added. The mixture was filtered and the organic layer washed with water, dried over Na₂SO₄, filtered and evaporated in vacuo to leave an oil. The oil was separated with flash chromatograpy (EtOAc) to leave the product as a white solid (1.17 g, 57.0%).

¹H-NMR (300 MHz, CDCl₃): δ 7.99-7.96 (m, 2H), 7.75-7.72 (m, 2H), 4.53-4.49 (m, 1H), 3.88-3.84 (m, 1H), 3.46-3.39 (m, 1H), 3.22-3.16 (m, 1H), 2.83-2.75 (m, 1H), 2.06 (s, 3H), 1.88-1.56 (m, 4

MS (ES): 279.0 [M+Na]+

### EXAMPLE 4

### Preparation of intermediate 4-(piperidin-4-ylcarbonyl)benzoic acid hydrochloride

A solution of (4-cyanophenyl) (1-acetylpiperidin-4-yl)methanone (1.17 g, 4.56 mmol) in conc. HCl (10 ml) was heated under reflux for 12 hrs. The reaction mixture was cooled to room temperature, filtered and the residue washed with a small amount of water. The solid was dried in vacuum to leave the product as a white solid (1.05 g, 86.0%).

¹H-NMR (300 MHz, DMSO-d₆): δ 13.35 (br s, 1H), 9.40 (br s, 1H), 9.15 (br s, 1H), 8.11-8.03 (m, 4H), 3.85-3.78 (m, 1H), 3.30-3.26 (m, 2H), 3-05-3.02 (m, 2H), 1.97-1.75 (m, 4H)

### EXAMPLE 5

### Preparation of intermediate methyl 4-(piperidin-4-ylcarbonyl)benzoate hydrochloride

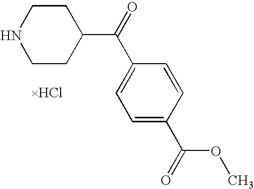

To a solution of 4-(piperidin-4-ylcarbonyl)benzoic acid hydrochloride in MeOH is added SOCl₂ at 0° C. The reaction mixture is heated to reflux, cooled to room temperature and evaporated in vacuo to leave the methyl ester as hydrochloride salt.

### EXAMPLE 6

### Synthesis of Methyl Ester Derivative of Ketanserin

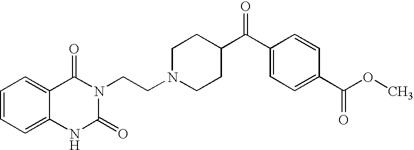

To a suspension of 3-(2-chloroethyl)-2,4(1H,3H)-quinazolinedione and K₂CO₃ in acetone is added methyl 4-(piperidin-4-ylcarbonyl)benzoate hydrochloride from Example 5 and the mixture is heated under reflux. The reaction mixture is cooled to room temperature, filtered and the filtrate evaporated in vacuo. The residue is separated with flash chromatography to leave the title compound.

### EXAMPLE 7

### Synthesis of Free Acid Derivative of Ketanserin

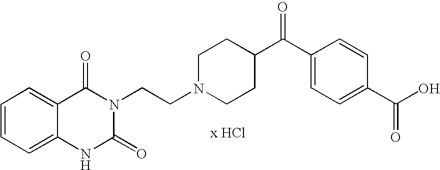

The methyl ester from Example 6 is added to a mixture of aqueous 2 M NaOH and MeOH and heated under reflux. The reaction mixture is cooled to 0° C. and aqueous 1 M HCl is added. The hydrochloride salt is precipitated out of the solution, filtered off and dried in vacuum to leave the title compound as a solid.

### EXAMPLE 8

### Preparation of intermediate 3-(1,2,3,6-tetrahydropyridin-4-yl)-5-methoxy-1H-indole

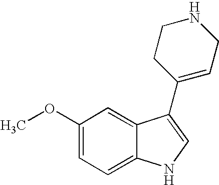

5-Methoxyindole (2.50 g, 17.0 mmol) and 4-piperidone hydrate hydrochloride (7.83 g, 51.0 mmol) was added to a solution of KOH (4.30 g, 76.5 mmol) in EtOH (85 ml) at room temperature and heated to reflux overnight. The reaction mixture was cooled to room temperature, filtered and the filtrate evaporated in vacuo. The residue was separated with flash chromatography (EtOAc/MeOH/NH₃, 7:3:0.5) to leave the product as a yellow solid (3.44 g, 88.8%).

¹H-NMR (300 MHz, DMSO-d₆): δ 11.10 (br s, 1H), 7.36-7.25 (m, 3H), 6.79-6.75 (m, 1H), 6.11 (t, 1H), 3.77 (s, 3H), 2.53 (br s, 2H), 3.04 (t 2H), 2.52-2.48 (m, 2H)

### EXAMPLE 9

### Alkylation of 3-(1,2,3,6-tetrahydropyridin-4-yl)-5-methoxy-1H-indole with methyl (4-bromomethyl)benzoate

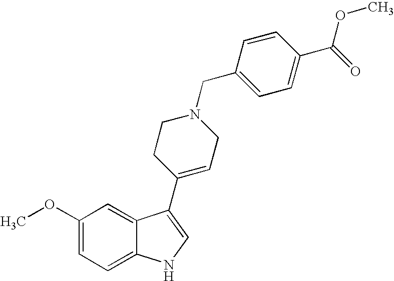

Following the procedure outlined in Example 6, 3-(1,2,3,6-tetrahydropyridin-4-yl)-5-methoxy-1H-indole was converted to the title compound as a yellow solid (0.98 g, 65.2%).

¹H-NMR (300 MHz, DMSO-d₆): δ 10.96 (br s, 1H), 7.94 (d, 2H), 7.51 (d, 2H), 7.33-7.24 (m, 3H), 6.78-6.74 (m, 1H), 6.07 (t, 1H), 3.85 (s, 3H), 3.75 (s, 3H), 3.65 (s, 2H), 3.12 (br s, 2H), 2.66 (t, 2H), 2.53-2.47 (m, 2H)

### EXAMPLE 10

### Preparation of intermediate 2,2,2-trichloroethyl 4-bromobutyrate

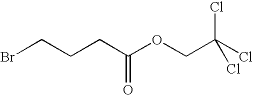

To a stirred solution of 4-bromobutyric acid (3.34 g, 20.0 mmol) in toluene (50 ml) was added 2,2,2-trichloroethanol 14.94 g, 0.10 mol) and p-toluenesulfonic acid monohydrate (7.60 g, 40.0 mmol) and the mixture refluxed with a Dean-Stark trap attached for 6 h. Water was removed continuously. The reaction mixture was cooled to room temperature and concentrated in vacuo. The mixture was added to CH₂Cl₂ (75 ml) and washed with H₂O (3×25 ml). The organic layer was dried over Na₂SO₄, filtered and evaporated in vacuo to leave an oil. The residue was distilled to leave the title compound as a colourless oil (4.77 g, 79.9%) (bp 100° C. at 0.5 mmHg).

¹H-NMR (300 MHz, CDCl₃): δ 4.74 (s, 2H), 3.48 (t, 2H), 2.65 (t, 2H), 2.21-2.13 (m, 2H)

### EXAMPLE 11

### Preparation of intermediate 4-(4-hydroxymethyl-piperidin-1-yl) butyric acid 2,2,2-trichloroethylester

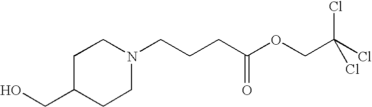

To a stirred solution of 4-piperidinemethanol (1.72 g, 15.0 mmol) in acetone (100 ml) was added K₂CO₃ (4.14 g, 30. mmol) and 2,2,2-trichloroethyl 4-bromobutyrate (4.47 g, 15.0 mmol) and the mixture was heated under reflux for 3 h. The reaction mixture was cooled to room temperature, filtered and the filtrate concentrated in vacuo. The residue was added to CH₂Cl₂ (75 ml) and washed with brine (25 ml) and H₂O (2×25 ml). The organic layer was dried over Na₂SO₄, filtered and evaporated in vacuo to leave the title compound as a viscous oil (4.70 g, 94.1%).

¹H-NMR (300 MHz, CDCl₃): δ 4.74 (s, 2H), 3.50 (d, 2H), 2.92 (d, 2H), 2.52-2.35 (m, 4H), 1.97-1.70 (m, 7H), 1.52-1.45 (m, 1H), 1.32-1.23 (m, 2H)

### EXAMPLE 12

### Synthesis of 1H-indole-3-carboxylic acid 1-[3-(2,2,2-trichloroethyl-ethoxycarbonyl)-propyl]-piperidine-4-ylmethyl ester

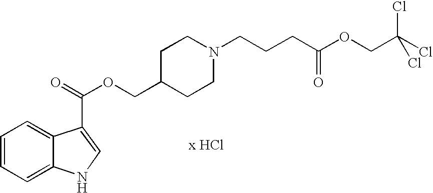

A suspension of indole-3-carboxylic acid (2.90 g, 18.0 mmol) in CH₂Cl₂ (75 ml) was treated with oxalyl chloride (1.84 ml, 20.7 mmol) and DMF (1 drop) and the mixture was stirred at room temperature for 2 h, then concentrated in vacuo to leave the acid chloride as a yellow solid. This was dissolved in a mixture of CH₂Cl₂ (30 ml) and THF (10 ml) and added dropwise (30 min) to a stirred solution of 4-(4-hydroxymethyl-piperidin-1-yl) butyric acid 2,2,2-trichloroethyl ester (from Example 11) (4.98 g, 15.0 mmol) and NEt₃ (1.82 g, 18.0 mmol) in CH₂Cl₂ (30 ml). The reaction mixture was stirred at room temperature overnight, treated with an aqueous satd. NaCl solution (25 ml) and 10% aqueous NaHCO₃ solution (25 ml). The organic layer was dried over Na₂SO₄, filtered and evaporated in vacuo to a brown viscous oil. The residue was separated with flash chromatography (SiO₂, EtOAc). The product was obtained as a pale yellow solid (1.83 g, 25.6%). Conversion to the hydrochloride salt was effected using etheral HCl.

¹H-NMR (300 MHz, CDCl₃): δ 9.02 (br s, 1H), 8.22-8.18 (m, 1H), 7.92 (d, 1H), 7.48-7.41 (m, 1H), 7.35-7.28 (m, 2H), 4.77 (s, 2H), 4.24 (d, 2H) 3.03 (d, 2H), 2.59-2.44 (q, 5H), 2.13-1.85 (m, 7H), 1.60-1.43 (m, 2H)

¹³C-NMR (75 MHz, CDCl₃): δ 171.7, 165.5, 136.2, 131.5, 125.7, 122.8, 121.7, 121.0, 111.7, 108.0, 94.8, 73.7, 67.9, 57.5, 53.1, 35.4, 31.7, 28.8, 21.8

MS (ES): 477.1 [M+H]⁺

### EXAMPLE 13

### Synthesis of 1H-indole-3-carboxylic acid 1-(3-carboxy-propyl)-piperidin-4-ylmethyl ester

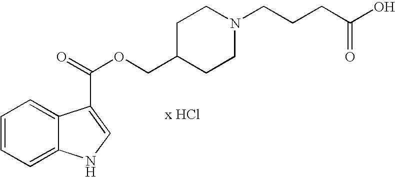

1H-indole-3-carboxylic acid 1-[3-(2,2,2-trichloroethyl-ethoxycarbonyl)-propyl] piperidine-4-ylmethyl ester (0.48 g, 1.0 mmol) was dissolved in a mixture of THF (25 ml) and aqueous 1 M KH₂PO₄(5 ml). Zn-powder (0.66 g, 10.0 mmol) was added and the resulting mixture stirred at room temperature for 24 h. The reaction mixture was filtered through a pad of kieselguhr and the filtrate evaporated in vacuo. The residue was separated with flash chromatography (SiO₂, EtOAc/MeOH (2:1)). The expected product was obtained as a white solid (0.29 g, 84.2%). Conversion to the hydrochloride salt was effected using etheral HCl.

¹H-NMR (300 MHz, DMSO): δ 11.98 (s, 1H), 8.08-7.97 (m, 2H), 7.47 (d, 1H), 7.20-7.17 (m 2H), 4.11 (d, 2H), 2.96 (d, 2H), 2.50-2.37 (m, 4H), 2.05 (t, 2H), 1.77-1.66 (m, 6H), 1.42-1.35 (m, 2H)

¹³C-NMR (75 MHz, DMSO): δ 171.7, 165.5, 136.2, 131.5, 125.7, 122.8, 121.7, 121.0, 20 111.7, 108.0, 94.8, 73.7, 67.9, 57.5, 53.1, 35.4, 31.7, 28.8, 21.8

MS (ES): 345.2 [M+H]⁺

### EXAMPLE 14

### Preparation of intermediate N-(1-benzylpiperidin-4-yl) napth-1-yl carboxamide

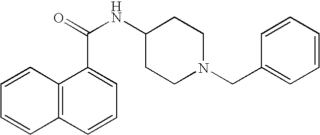

To a stirred suspension of 1-napthoic acid (8.61 g, 0.050 mol) in CH₂Cl₂ (150 ml) was added SOCl₂ (23.79 g, 0.20 mol) and the mixture was heated under reflux for 4 h. The mixture was evaporated in vacuo to leave the acid chloride as a solid material. This was dissolved in CH₂Cl₂ (150 ml) and added dropwise to a stirred solution of 4-amino-1-benzylpiperidine (9.51 g, 0.050 mol) and NEt₃ (5.06 g, 0.05 mol) in CH₂Cl₂ (100 ml) at 0° C. The mixture was stirred at room temperature for 24 h and washed with H₂O (3×75 ml) The organic layer was dried over Na₂SO₄ and evaporated in vacuo to a solid material. This was recrystallized from ethanol/water (40/60) to leave the product as a white solid (7.8 g, 45.3%).

¹H-NMR (300 MHz, CDCl₃): δ 8.32-8.27 (m, 1H), 7.90 (t, 2H), 7.57-7.30 (m, 9H), 6.17 (d, 2H), 4.17-4.06 (m, 1H), 3.55 (s, 2H), 2.88 (d, 2H), 2.27-2.05 (m, 4H), 1.69-1.50 (m, 2H)

### EXAMPLE 15

### Preparation of intermediate N-(piperidin-4-yl)napth-1-yl carboxamide hydrochloride

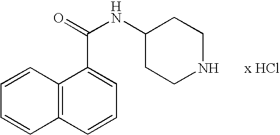

A solution of N-(1-benzylpiperidin-4-yl)napth-1-yl carboxamide (1.38 g, 4.0 mmol) in dry CH₂Cl₂ (15 ml) was cooled to 0° C., α-chloroethyl chloroformate (1.14 g, 8.0 mmol) was added and the mixture was stirred for 30 minutes. The mixture was evaporated in vacuo, MeOH (15 ml) was added and the mixture was heated under reflux for 1 h. The reaction mixture was evaporated in vacuo and the residue recrystallized from acetonitrile to give the product as a white powder (1.01 g, 86.8%).

¹H-NMR (300 MHz, CDCl₃): δ 8.32-8.27 (m, 1H), 7.90 (t, 2H), 7.57-7.30 (m, 9H), 6.17 (d, 2H), 4.17-4.06 (m, 1H), 3.55 (s, 2H), 2.88 (d, 2H), 2.27-2.05 (m, 4H), 1.69-1.50 (m, 2H)

### EXAMPLE 16

### Alkylation of N-(piperidin-4-yl)napth-1-yl carboxamide hydrochloride with 2,2,2-trichloroethyl 4-bromobutyrate

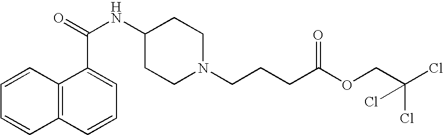

To a stirred suspension of N-(piperidin-4-yl)napth-1-yl carboxamide hydrochloride (0.58 g, 2.0 mmol) in acetone (20 ml) was added K₂CO₃ (1.10 g, 8.0 mmol) and 2,2,2-trichloroethyl 4-bromobutyrate (0.89 g, 3.0 mmol) and the mixture was heated under reflux for 24 h. The mixture was cooled to room temperature and filtered. The filtrate was evaporated in vacuo and to the residue was added CH₂Cl₂ (50 ml). It was then washed with H₂O (3×25 ml). The organic layer was dried over Na₂SO₄, filtered and evaporated in vacuo to an oil. The oil was separated with flash chromatography (SiO₂, EtOAc/MeOH (1:1)) to give the product as a white solid (0.87 g, 92.2%).

¹H-NMR (200 MHz, CDCl₃): δ 8.26-8.21 (m, 1H), 7.83 (t, 2H), 7.56-7.37 (m, 4H), 5.89 (d, 2H), 4.71 (s, 2H), 4.17-4.06 (m, 1H), 2.84 (d, 2H), 2.49 (t, 2H), 2.39 (t, 2H), 2.21-2.11 (m, 4H), 1.93-1.82 (p, 2H), 1.62-1.49 (m, 2H)

¹³C-NMR (75 MHz, CDCl₃): δ 173.0, 168.4, 134.1, 133.0, 129.8, 129.4, 127.7, 126.4, 20 125.6, 124.8, 124.2, 124.1, 73.8, 60.0, 57.0, 52.0, 46.5, 31.6, 22.0, 14.1

MS (ES): 494.2 [M+Na]⁺

### EXAMPLE 17

### Hydrolysis of the Trichloroethyl Ester from Example 16

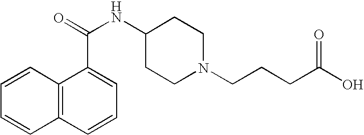

Following the procedure outlined in Example 13, the trichloroethyl ester from Example 16 (0.67 g, 1.4 mmol) was converted to the title compound as a white solid (0.38 g, 79.6%).

¹H-NMR (200 MHz, DMSO-d₆): δ 8.50 (d, 2H), 8.39-8.18 (m, 1H), 8.04-7.96 (m, 2H), 7.62-7.50 (m, 4H), 3.91 (br s, 1H), 2.93 (d, 2H), 2.37 (t, 2H), 2.23-2.06 (m, 4H), 1.92 (d, 2H), 1.72-1.57 (m, 4H)

¹³C-NMR (50 MHz, DMSO-D₆): δ 167.9, 135.1, 133.0, 129.7, 129.5, 128.1, 126.6, 126.1, 125.3, 125.0, 124.9, 57.4, 52.0, 46.6, 38.6, 33.5, 31.2, 22.2

MS (ES): 363.1 [M+Na]⁺

### EXAMPLE 18

### Alkylation of N-(piperidin-4-yl)napth-1-yl carboxamide hydrochloride with ethyl 4-bromobutyrate

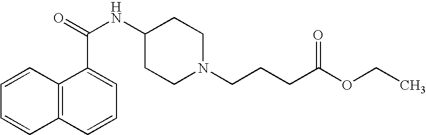

Following the procedure outlined in Example 16, N-(piperidin-4-yl)napth-1-yl carboxamide hydrochloride (0.58 g, 2.0 mmol) was converted to the title compound as a white solid (0.67 g, 91.4%).

¹H-NMR (300 MHz, CDCl₃): δ 8.24 (d, 1H), 7.89-7.82 (m, 2H), 7.54-7.49 (m, 3H), 7.40 (t, 1H), 6.07 (d, 2H), (m, 3H), 2.84 (d, 2H), 2.36-2.28 (m, 4H), (m, 4H), 1.81-1.76 (p, 2H), 1.55-1.51 (m, 2H), 1.26 (t, 3H)

¹³C-NMR (75 MHz, CDCl₃): δ 173.3, 168.7, 134.5, 133.4, 130.2, 129.8, 128.1, 126.8, 126.1, 125.1, 124.6, 124.5, 60.1, 57.4, 52.1, 46.9, 32.0, 22.2, 14.0

MS (ES): 391.2 [M+Na]⁺

### EXAMPLE 19

### Preparation of intermediate (piperidin-4-yl) ethylcarboxylate hydrochloride

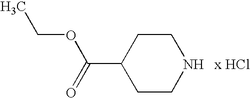

A stirred solution of isonipectoic acid (12.9 g, 0.10 mol) in absolute ethanol (200 ml) was cooled to 0° C. and SOCl₂ (47.5 g, 0.40 mol) was added dropwise. The mixture was stirred at room temperature and heated to reflux for 3 h. The reaction mixture was evaporated in vacuo and the residue dissolved in a 10% aqueous solution of NaOH (250 ml). The aqueous solution was extracted with CH₂Cl₂ (3×100 ml). The organic extract was dried over NaSO₄, filtered and evaporated in vacuo. The residue was dissolved in dry ethanol and HCl was bubbled into the solution to give the hydrochloride precipitate. The residue was recrystallized from absolute ethanol to give the product as a white solid (17.46 g, 90.2%).

¹H-NMR (300 MHz, CDCl₃): δ 9.40 (br s, 2H), 4.09-4.02 (q, 2H), 3.30 (d, 2H), 3.01-2.95 (m, 2H), 2.56-2.47 (m, 1H), 2.14-1.95 (m, 4H), 1.30 (t, 3H)

### EXAMPLE 20

### Preparation of intermediate (1-benzylpiperidin-4-yl)ethylcarboxylate hydrochloride

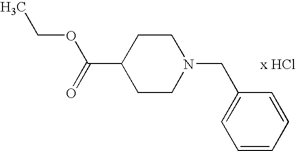

To a suspension of (piperidin-4-yl)ethylcarboxylate hydrochloride (8.6 g, 44.4 mmol) and K₂CO₃ (24.5 g, 0.17 mol) in acetone (200 ml) was added benzylbromide (9.11 g, 53.3 mmol) and the mixture was heated to reflux for 12 h. The solvent was evaporated in vacuo and to the residue was added H₂O (200 ml). The aqueous layer was extracted with Et₂O (3×100 ml) and the organic extracts dried over Na₂SO₄, filtered and evaporated in vacuo. The residue was dissolved in acetone and HCl was bubbled into the solution to give the hydrochloride precipitate. The precipitate was filtered, dried and recrystallized from acetone to give the expected product as a white solid (11.03 g, 87.6%).

¹H-NMR (300 MHz, DMSO-d₆): δ 11.48 (br s, 1H), 7.67 (s, 2H), 7.42 (s, 3H), 4.30-4.25 (m, 2H), 4.11-4.01 (m, 2H), 3.28 (d, 2H), 2.97-2.84 (m, 2H), 2.15-1.99 (m, 4H) 1.15 (t, 3H)

### EXAMPLE 21

### Preparation of intermediate 1-[(1-benzylpiperidin-4-yl)methanol

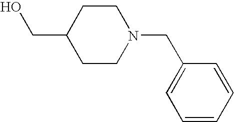

A suspension of LiAlH₄ (1.52 g, 40.0 mmol) in dry THF (30 ml) was stirred at 0° C. and a solution of 1-benzylpiperidin-4-yl)ethylcarboxylate hydrochloride (2.47 g, 10.0 mmol) in dry THF (50 ml) was added dropwise. The obtained mixture was heated under reflux for 4 h and then cooled to room temperature. EtOAc (200 ml), water (40 ml), and a 2 N aqueous solution of NaOH (10 ml) were added. The obtained mineral precipitate was filtered through a pad of kieselguhr, the filtrate evaporated in vacuo and water (50 ml) added to the residue. The aqueous layer was extracted with CH₂Cl₂ (3×50 ml) and the organic extracts were combined and dried over Na₂SO₄, filtered and evaporated in vacuo to give the product as a colourless oil (1.75 g, 85.6

¹H-NMR (200 MHz, CDCl₃): δ 7.40-7.26 (m, 5H), 3.53-3.46 (m, 4H), 2.94 (d, 2H), 2.61 (br s, 1H), 2.00 (t, 2H), 1.75 (d, 2H), 1.48-1.26 (m, 3H)

### EXAMPLE 22

### Synthesis of 1-[(1-benzylpiperidin-4-yl)carboxymethyl] napthalene

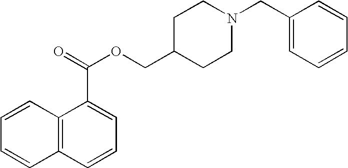

A stirred solution of (1-benzylpiperidin-4-yl)methanol (2.79 g, 13.7 mmol) and NEt₃ (1.65 g, 16.3 mmol) in CH₂Cl₂ (50 ml) was cooled to 0° C. and a solution of napthoyl chloride (prepared as in Example 16) (3.11 g, 16.3 mmol) dissolved in CH₂Cl₂/THF (1:1, 50 ml) was added dropwise. The resulting mixture was stirred at room temperature overnight, evaporated in vacuo and to the residue was added EtOAc (100 ml). The organic layer was washed with water (50 ml), brine (50 ml) and water (50 ml). The organic layer was dried over Na₂SO₄, filtered and evaporated in vacuo to give an oil. The oil was separated with flash chromatography (SiO₂, EtOAc) to give the expected product as a yellow oil (3.12 g, 63.3%).

¹H-NMR (300 MHz, CDCl₃): δ 8.94 (d, 1H), 8.22-8.19 (m, 1H), 8.04 (d, 1H), 7.90 (d, 1H), 7.64-7.49 (m, 3H), 7.49-7.25 (m, 5H), 4.30 (d, 2H), 3.52 (s, 2H), 2.98 (d, 2H), 2.09-2.00 (m, 2H), 1.93-1.83 (m, 3H), 1.57-1.45 (m, 2H)

MS (ES): 360.1 [M+H]⁺

### EXAMPLE 23

### Preparation of intermediate 1-[(piperidin-4-yl) methyloxycarbonyl]napthalene hydrochloride

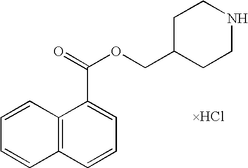

Following the procedure outlined in Example 15, 1-[(1-benzylpiperidin-4-yl)carboxymethyl]napthalene (1.69 g, 4.70 mml) was converted to the title compound as a yellow solid (1.07 g, 74.5%).

¹HH-NMR (300 MHz, CDCl₃): δ 9.60 (br s, 2H), 8.86 (d, 1H), 8.17 (d, 1H), 8.00 (d, 1H), 7.85 (d, 1H), 7.61-7.34 (m, 3H), 4.28 (d, 2 HO, 3.55 (d, 2H), 2.89 (d, 2H), 2.04-1.68 (m, 5H)

### EXAMPLE 24

### Alkylation of 1-[(piperidin-4-yl)methyloxycarbonyl] napthalene hydrochloride with 2,2,2-trichloroethyl 4-bromobutyrate

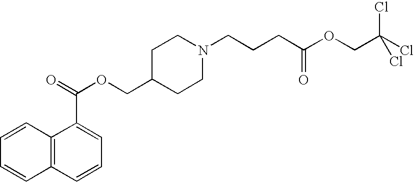

Following the procedure outlined in Example 16, 1-[(piperidin-4-yl)methyloxycarbonyl]napthalene hydrochloride (0.30 g, 1.0 mmol) was converted to the title compound as a white solid (0.43 g, 89.5%).

¹H-NMR (300 MHz, CDCl₃): δ 8.89 (d, 1H), 8.18-8.14 (m, 1H), 8.00 (d, 1H), 7.88-7.84 (m, 1H), 7.63-7.43 (m, 3H), 4.72 (s, 2H), 4.24 (d, 2H), 2.93 (d, 2H), 2.49 (t, 2H), 2.38 (t, 2H), 1.96-1.79 (m, 8H), 1.46-1.27 (m, 2H)

¹³C-NMR (75 MHz, CDCl₃): δ 171.9, 167.5, 133.8, 133.2, 131.3, 130.0, 128.5, 127.7, 127.2, 126.1, 125.7, 124.4, 95.0, 73.9, 69.3, 60.3, 57.7, 53.3, 35.5, 31.9, 29.1, 22.1, 21.0, 14.1

MS (ES): 487.1 [M+H]⁺

### EXAMPLE 25

### Alkylation of 1-[(piperidin-4-yl)methyloxycarbonyl] napthalene hydrochloride with ethyl 4-bromobutyrate

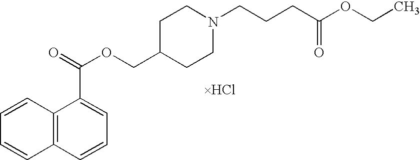

Following the procedure outlined in Example 16, 1-[(piperidin-4-yl)methyloxycarbonyl]napthalene hydrochloride (0.39 g, 1.27 mmol) was converted to the title compound as a yellow oil. The oil was dissolved in Et₂O and HCl was bubbled into the solution to give the hydrochloride precipitate. The precipitate was filtered off, dried and recrystallized from acetonitrile to leave the hydrochloride salt (0.31 g, 73.8%).

¹H-NMR (300 MHz, DMSO-d₆): δ 10.7 (br s, 1H), 8.76 (d, 1H), 8.23 (t, 2H), 8.04 (d, 1H), 7.71-7.59 (m, 3H), 4.28 (d, 2H), 4.11-4.03 (q, 2H), 3.49 (d, 2H), 3.51-2.96 (m, 5H), 10 2.41 (t, 2H), 2.00-1.83 (m, 7H), 1.19 (t, 3H)

¹³C-NMR (75 MHz, DMSO-d₆): δ 172.8, 167.4, 134.4, 134.2, 131.3, 131.0, 129.6, 128.8, 127.3, 127.2, 125.8, 125.7, 68.6, 60.9, 56.0, 52.0, 33.7, 31.4, 26.4, 19.6, 14.9

MS (ES): 406.2 [M+Na]⁺

### EXAMPLE 26

### Hydrolysis of the Trichloroethyl Ester from Example 25

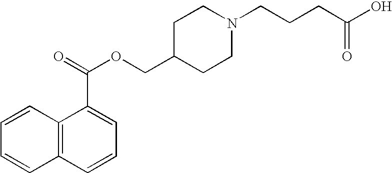

Following the procedure outlined in Example 13, the trichloroethyl ester from Example 25 (0.43 g, 0.88 mmol) was converted to the title compound as a white solid (0.25 g, 79.9%).

¹H-NMR (300 MHz, DMSO-d₆): δ 10.11, 8.75 (d, 1H), 8.15 (t, 2H), 8.01 (d, 1H), 7.68-7.57 (m, 3H), 4.20 (d, 2H), 2.90 (d, 2H), 2.33 (t, 2H), 2.20 (t, 2H), 2.03-1.92 (t, 2H), 1.75-1.60 (m, 5H), 1.45-1.30 (m, 2H)

¹³C-NMR (75 MHz, DMSO-d₆): δ 175.0, 166.6, 133.4, 133.3, 130.4, 129.8, 128.6, 127.8, 126.7, 126.3, 125.0, 124.8, 68.7, 57.4, 52.3, 34.7, 33.2, 28.0, 21.6

MS (ES): 378.1 [M+Na]⁺

### EXAMPLE 27

### Alkylation of 1-[(piperidin-4-yl)methyloxycarbonyl] napthalene hydrochloride with diethyl 2-bromoethylphosphonate

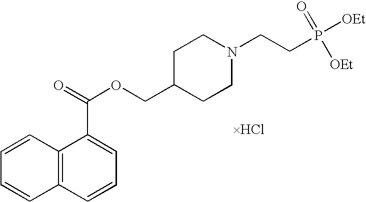

Following the procedure outlined in Example 16, 1-[(piperidin-4 yl)methyloxycarbonyl]-napthalene hydrochloride (0.63 g, 2.06 mmol) was converted to the title compound as a yellow oil. The oil was dissolved in Et₂O and HCl was bubbled into the solution to give a white precipitate. The precipitate was filtered off, dried and recrystallized from acetonitrile to leave the hydrochloride salt (0.37 g, 37.9%)

¹H-NMR (300 MHz, DMSO-_{d6}): δ 11.0 (br s, 1H), 8.78 (d, 1H), 8.28-8.21 (m, 2H), 8.09-8.05 (m, 1H), 4.30 (d, 2H), 4.14-4.03 (q, 4H), 3.63-3.52 (m, 2H), 3.20-2.95 (m, 5H), 2.48-2.37 (m, 1H), 2.02-1.81 (m, 5H), 1.28 (t, 6H)

¹³C-NMR (75 MHz, DMSO-_{d6}): δ 166.5, 133.4, 133.3, 130.4, 130.1, 128.7, 127.9, 126.4, 126.3, 124.9, 124.8, 67.7, 61.6, 61.5, 50.7, 50.0, 32.9, 26.5, 21.1, 19.3, 16.2, 16.1

MS (ES): 456.2 [M+Na]⁺

### EXAMPLE 28

### Preparation of intermediate N-(1-benzylpiperidin-4-yl)-indazole-3-carboxamide

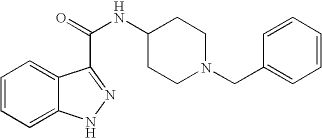

To a stirred solution of 1-H-indazole-3-carboxylic acid (8.11 g, 50.0 mmol) in dry DMF (140 ml) under argon atmosphere was added CDI (8.92 g, 55 mmol). The mixture was heated at 60° C. for 2 h. The mixture was cooled to room temperature, and 4-amino-1-benzylpiperidine (9.51 g, 50.0 mmol) previously dissolved in DMF (20 ml) was added dropwise. The mixture was heated at 60° C. for 2 h, cooled to room temperature and the solvent evaporated in vacuo. To the residue was added CH₂Cl₂ (250 ml) and the organic layer was washed with H₂O (100 ml), 1 N aqueous NaOH (100 ml), H₂O (100 ml) and brine (100 ml). The organic layer was dried over Na₂SO₄, filtered and evaporated in vacuo. The residue was recrystallized from EtOH to leave the expected product as a white solid (14.23 g, 85.1%).

¹H-NMR (200 MHz, DMSO-d₆): δ 13.59, 8.20 (t, 2H), 7.61 (t, 1H), 7.38-7.21 (m, 7H), 3.95-3.87 (m, 1H), 3.49 (s, 2H), 2.80 (d, 2H), 2.04 (t, 2H), 1.78-1.67 (4H)

### EXAMPLE 29

### Preparation of intermediate N-(1-benzylpiperidin-4-yl)-1-isopropylindazole-3-carboxamide

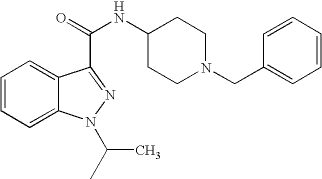

To a solution of N-(1-benzylpiperidin-4-yl)-indazole-3-carboxamide (3.34 g, 10.0 mmol) in dry DMF (70 ml) under argon atmosphere was added sodium hydride (0.25 g, 10.0 mmol) and the mixture was stirred at room temperature for 3 h. To the mixture was added isopropylbromide (1.37 g, 11.0 mmol) and stirring continued for an additional 24 h. The reaction mixture was evaporated in vacuo and to the residue was added EtOAc (100 ml). The organic layer was washed with brine (50 ml) and H₂O (2×50 ml). The organic layer was dried over Na₂SO₄, filtered and evaporated in vacuo to leave an oil that solidified upon standing. The oil was separated with flash chromatography (SiO₂, Et₂O/Hexane (2:1) to leave the product as a solid (1.22 g, 32.7%)

¹H-NMR (200 MHz, CDCl₃): δ 8.40 (d, 1H), 7.44-7.27 (m, 8H), 6.95 (d, 1H), 4.92-4.83 (p, 1H), 4.05-3.95 (m, 1H), 3.55 (s, 1H), 2.91 (d, 2H), 2.21 (t, 2H), 2.08 (d, 2H), 1.71-1.60 (m, 8H)

### EXAMPLE 30

### Preparation of intermediate N-(1-piperidin-4-yl)-1-isopropylindazole-3-carboxamide hydrochloride

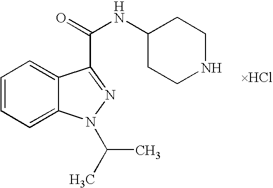

Following the procedure outlined in Example 15, N-(1-benzylpiperidin-4-yl)-1-isopropylindazole-3-carboxamide (1.77 g, 4.28 mmol) was converted to the title compound as a white solid (1.26 g, 91.1%).

¹H-NMR (200 MHz, CDCl₃): δ 9.80 (br s, 1H), 9.68 (br s, 1H), 8.34 (d, 1H), 7.49-7.31 (m, 2H), 7.28-7.26 (m, 1H), 7.05 (d, 1H), 4.94-4.85 (m, 1H), 4.35-4.32 (m, 1H), 3.62 (d, 2H), 3.15-3.04 (m, 2H), 2.36-2.32 (m, 1H), 2.18-2.08 (m, 3H), 1.65 (d, 6H)

### EXAMPLE 31

Alkylation of N-(1-piperidin-4-yl)-1-isopropylindazole-3-carboxamide hydrochloride with ethyl 4-bromobutyrate

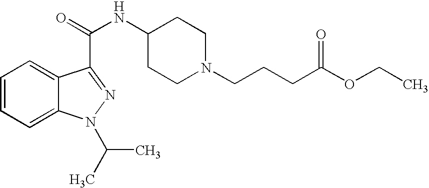

Following the procedure outlined in Example 16, N-(1-piperidin-4-yl)-1-isopropylindazole-3-carboxamide hydrochloride (0.32 g, 1.0 mmol) was converted to the title compound as a colourless oil (0.37 g, 93.7%).

¹H-NMR (300 MHz, CDCl₃): δ 8.38 (d, 1H), 7.43-7.37 (m, 3H), 7.26 (d, 1H), 4.90-4.82 (m, 1H), 4.19-4.08 (m, 1H), 2.91 (d, 2H), 2.43-2.31 (m, 4H), 2.19-2.03 (m, 5H), 1.87-1.58 (m, 11H), 1.26 (t, 3H)

¹³C-NMR (75 MHz, CDCl₃): δ 173.3, 162.0, 139.8, 136.7, 126.2, 122.8, 122.7, 122.2, 109.1, 60.1, 57.4, 52.3, 50.7, 46.0, 32.2, 32.1, 22.2, 21.9, 14.1

MS (ES): 423.1 [M+Na]⁺

Conversion to the hydrochloride salt was effected using ethereal HCl.

### EXAMPLE 32

### Alkylation of N-(1-piperidin-4-yl)-1-isopropylindazole-3-carboxamide hydrochloride with 2,2,2-trichloroethyl 4-bromobutyrate

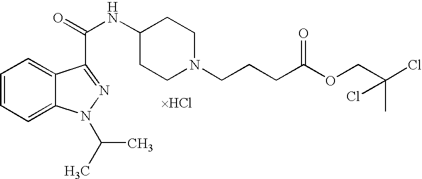

To a stirred suspension of N-(1-piperidin-4-yl)-1-isopropylindazole-3-carboxamide hydrochloride (0.32 g, 1.0 mmol) and K₂CO₃ (0.55 g, 4.0 mmol) in acetone (15 ml) was added 2,2,2-trichloroethyl 4-bromobutyrate (0.45 g, 1.5 mmol) and the mixture heated under reflux for 12 h. The mixture was cooled to room temperature, filtered and the filtrate evaporated in vacuo. To the residue was added EtOAc (30 ml) and the organic layer washed with H₂O (15 ml), brine (15 ml) and H₂O (15 ml). The organic layer was dried over Na₂SO₄, filtered and the solvent evaporated in vacuo to leave an oil. The oil was dissolved in acetone and 1.0 M HCl in Et₂O was added dropwise to give a white precipitate. The precipitate was filtered off, dried and recrystallized from acetone to leave the hydrochloride salt as a white powder (0.47 g, 87.0%).

¹H-NMR (300 MHz, CDCl₃): δ 12.44 (br s, 1H), 8.29 (d, 1H), 7.47-7.37 (m, 2H), 7.25 (t, 1H), 7.16 (d, 1H), 4.89-4.85 (m, 1H), 4.76 (s, 2H), 4.19-4.08 (m, 1H), 3.71 (d, 2H), 3.14-2.69 (m, 4H), 2.34-2.03 (m, 8H), 1.61 (d, 6H)

¹³C-NMR (75 MHz, CDCl₃): δ 170.3, 162.4, 139.9, 135.9, 126.4, 122.8, 122.6, 122.3, 109.3, 94.6, 74.0, 56.2, 52.2, 50.9, 43.8, 30.7, 29.1, 22.0, 18.9, 15.2

MS (ES): 526.2 [M+Na]+

### EXAMPLE 33

### Hydrolysis of the Trichloroethyl Ester from Example 32

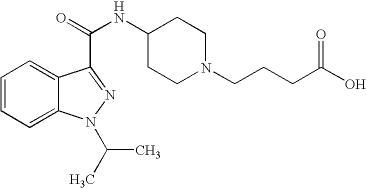

Following the technique outlined in Example 13, the trichloroethyl ester from Example 32 (0.37 g, 0.69 mmol) was converted to the title compound as a white solid (0.20 g, 77.9%)

¹H-NMR (300 MHz, CD₃OD): δ 8.21 (d, 1H), 7.61 (d, 1H), 7.42-7-37 (m, 1H), 7.24 (t, 1H), 5.01-4.93 (m, 1H), 4.24-4.19 (m, 1H), 3.51 (d, 2H), 3.05-2.91 (m, 4H), 2.45 (t, 2H), 2.18-2.14 (m, 2H), 1.99-1.88 (m, 4H), 1.57 (d, 6H)

¹³C-NMR (75 MHz, CD₃OD): δ 181.1, 164.5, 141.4, 137.6, 127.6, 124.0, 123.7, 123.0, 110.9, 59.1, 52.6, 52.1, 45.6, 37.4, 30.5, 22.3, 21.7

MS (ES): 395.1 [M+Na]⁺

### EXAMPLE 34

### Preparation of intermediate 4-bromomethyl benzoic acid 2,2,2-trichloroethyl ester

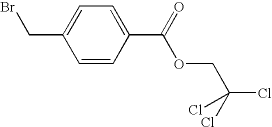

To a solution of 2,2,2-trichloroethanol (2.46 g, 16.5 mmol) and NEt₃ (1.67 g, 16.5 mmol) in CH₂Cl₂ (40 ml) at 0° C. was added dropwise 4-bromomethyl benzoylbromide (4.17 g, 15.0 mmol) in CH₂Cl₂ (20 ml) and the mixture was stirred at room temperature overnight. To the reaction mixture was added H₂O (20 ml) and the organic layer was separated. The organic layer was washed with aqueous 1 M HCl (20 ml) and H₂O (20 ml). The organic layer was dried over Na₂SO₄, filtered and the solvent evaporated in vacuo to leave the expected product as a white solid.

¹H-NMR (300 MHz, CDCl₃): δ 7.86 (dd, 4H), 5.00 (s, 2H), 4.54 (s, 2H)

### EXAMPLE 35

### Alkylation of N-(1-piperidin-4-yl)-1-isopropylindazole-3-carboxamide hydrochloride with 4-bromomethyl benzoic acid 2,2,2-trichloroethyl ester

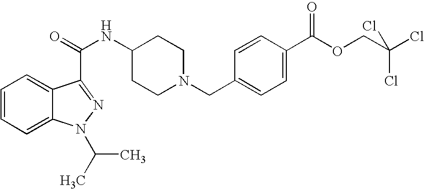

Following the procedure outlined in Example 16, N—)1-(piperidin-4-yl)-1-isopropylindazole-3-carboxamide hydrochloride (0.41 g, 1.3 mmol) was converted to the title compound as a colourless oil (0.61 g, 85.2%).

¹H-NMR, (300 MHz, CDCl₃): δ 8.38 (d, 1H), 8.09 (d, 2H), 7.50-7.36 (m, 4H), 7.29-7.23 (m, 1H), 6.95 (d, 1H) 4.98 (s, 2H), 4.92-4.83 (p, 1H), 4.13-4.04 (m, 1H), 3.60 (s, 2H), 2.88 (d, 2H), 2.24 (t, 2H), 2.10-2.05 (m, 2H), 1.75-1.61 (m, 8H)

¹³C-NMR (75 MHz, CDCl₃): δ 165.2, 162.6, 145.9, 140.3, 137.3, 130.5, 129.3, 128.1, 127.8, 126.7, 123.4, 123.3, 122.8, 109.8, 95.6, 74.7, 63.0, 53.0, 51.3, 46.4, 32.8, 22.5

MS (ES): 551.1 [M+H]⁺

### EXAMPLE 36

### Hydrolysis of the Trichloroethyl Ester from Example 35

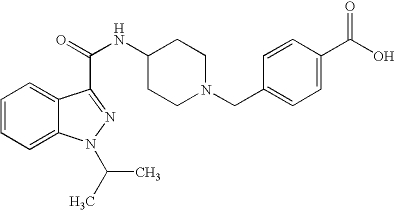

Following the procedure outlined in Example 13, the trichloroethyl ester from Example 35 (0.42 g, 0.76 mmol) was converted to the title compound as a white solid (0.25 g, 78.9%).

¹H-NMR (300 MHz, CD₃OD): δ 8.22 (d, 1H), 8.04 (d, 1H), 7.66 (d, 1H), 7.48-7.41 (m, 3H), 7.26 (t, 1H), 5.08-4.99 (m, 1H), 4.10-4.03 (m, 1H), 3.13 (d, 2H), 2.53 (t, 2H), 2.08-2.04 (m, 2H), 1.92-1.85 (m, 2H), 1.61 (d, 2H)

¹³C-NMR (75 MHz, CD₃OD): δ 163.5, 140.4, 136.7, 129.7, 126.6, 123.0, 122.6, 122.0, 109.9, 61.7, 52.2, 51.1, 46.0, 30.6, 21.3

MS (ES): 419.1 [M+H]⁺

### EXAMPLE 37

### Preparation of intermediate 4-aminomethyl-1-(tert-butoxycarbonyl)piperidine

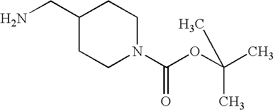

Benzaldehyde (8.73 g, 82.3 mmol) was added all at once to a stirred solution of 4-minomethylpiperidine (9.42 g, 82.3 mmol) in toluene (100 ml). The mixture was heated under reflux for 4 h with a Dean-Stark trap attached to collect the water. The reaction mixture was cooled to room temperature and di-tert-butyldicarbonate (19.75 g, 90.5 mmol) was added in divided portions under continuous stirring. The mixture was stirred overnight, evaporated in vacuo and the residue stirred vigorously with aqueous 1 N KHSO₄ (100 ml) at room temperature for 4 h. The mixture was extracted with Et₂O (3×100 ml) and then the aqueous layer was made strongly basic with NaOH. The aqueous layer was extracted with CH₂Cl₂ (3×100 ml). The combined extracts were dried with Na₂SO₄, filtered and the solvent evaporated in vacuo to leave the product as an oil (15.4 g, 86.5%)

¹H-NMR (200 MHz, DMSO-_{d6}): δ 4.04-4.01 (m, 2H), 2.60 (t, 2H), 2.50 (d, 2H), 1.62 (d, 2H), 1.32 (s, 9H), 1.31-1.28 (m, 1H), 1.06 (br s, 2H), 1.03-0.93 (m, 2H)

### EXAMPLE 38

### Synthesis of 4-amino-N-(tert-butoxycarbonyl) peridin-4-ylmethyl]-5-chloro-2-methoxybenzamide

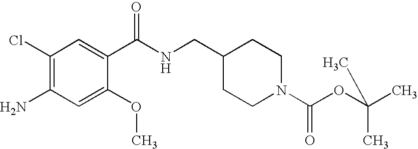

To a mixture of 4-aminomethyl-1-(tert-butoxycarbonyl)piperidine (10.0 g, 46.7 mmol), 4-amino-5-chloro-2-methoxybenzoic acid (9.41 g, 46.7 mmol) and NEt₃ (6.80 ml, 46.7 mmol) in DMF (100 ml) were added 1-ethyl-3-[3-(dimethylamino)propyl]carbodiimide hydrochloride (EDC) g, 46.7 mmol) and 1-hydroxybenzotriazole (HOBT) g, 46.7 mmol) at 0° C. The reaction mixture was stirred at room temperature overnight and concentrated in vacuo. The resulting residue was added H₂O (100 ml) and extracted with EtOAc. The combined organic extracts were washed with aqueous K₂CO₃ and dried over Na₂SO₄. The solvent was removed in vacuo and the residue separated with flash chromatography (SiO₂, EtOAc) to give the expected product as a white solid (11.91 g, 64.1%).

¹H-NMR (200 MHz, CDCl₃): δ 8.06 (s, 1H), 7.77 (t, 1H), (s, 1H), 4.64 (s, 2H), 4.08 (d, 2H), 3.86 (s, 3 H), 3.30 (t, 2H), 2.67 (t, 2H), 1.78-1.66 (m, 3H), 1.43 (s, 9H), 1.24-1.11 (m, 2H)

### EXAMPLE 39

### Preparation of intermediate 4-amino-5-chloro-2-methoxy-N-(piperidin-4-ylmethyl)benzamide hydrochloride

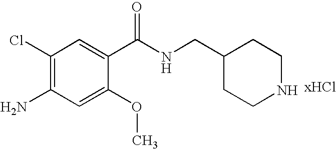

To a stirred solution of 4-amino-N-(tert-butoxycarbonyl)piperidin-4-ylmethyl]-5-chloro-2-methoxybenzamide (1.70 g, 4.3 mmol) in 1,4-dioxane (30 ml) at 0° C. was added 4 M HCl in 1,4-dioxane (10 ml) in portions. The reaction mixture was stirred at room temperature for 4 h, evaporated in vacuo and the residue recrystallized from acetone to leave the product as a red solid (0.89 g, 61.8%).

¹H-NMR (300 MHz, DMSO-D₆): δ 9.28 (br s, 1H), 9.04 (br s, 1H), 8.00 (t, 1H), 7.64 (s, 1H), 7.31 (br s, 4H), (s, 1H), 3.81 (s, 3H), 3.21-3.16 (m, 4H), 2.82-2.71 (q, 2H), 1.80-1.71 (m, 3H), 1.45-0.34 (m, 2H)

### EXAMPLE 40

### Alkylation of 4-amino-5-chloro-2-methoxy-N-(piperidin-4-ylmethyl)benzamide hydrochloride with ethyl 4-bromobutyrate

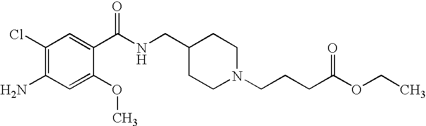

Following the method outlined in Example 16, 4-amino-5-chloro-2-methoxy-N-(piperidin-4-ylmethyl)benzamide hydrochloride (0.76 g, 1.98 mmol) was converted to the title compound as an oil (0.57 g, 69.9%).

¹H-NMR (300 MHz, CDCl₃): δ 8.08 (S, 1H), 7.74 (t, 1H), 6.30 (S, 1H), 4.49 (s, 2H), 4.14-4.07 (q, 2H), 3.87 (s, 3H), 3.30 (t, 2H), 2.90 (d, 2H), 2.35-2.28 (m, 4H), 1.94-1.69 (m, 7H), 1.29-1.18 (m, 5H)

¹³C-NMR (75 MHz, CDCl₃): δ 173.9, 164.9, 157.7, 147.0, 133.4, 112.9, 111.9, 98.2, 66.2, 58.3, 56.5, 53.8, 45.5, 36.5, 32.7, 30.4, 15.6

MS (ES): 411.9 [M+H]⁺

Conversion to the hydrochloride salt was effected with ethereal HCl.

### EXAMPLE 41

### Alkylation of 4-amino-5-chloro-2-methoxy-N-(piperidin-4-ylmethyl)benzamide hydrochloride with diethyl 2-bromoethylphosphonate

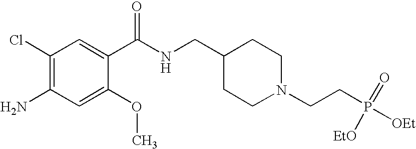

Following the procedure outlined in Example 16, 4-amino-5-chloro-2-methoxy-N-(piperidin-4-ylmethyl)benzamide hydrochloride (1.53 g, 4.0 mmol) was converted to the title compound as an oil (1.12 g, 57.1%).

¹H-NMR (300 MHz, CDCl₃): δ 7.99 (S, 1H), 7.70 (t, 1H), 6.29 (s, 1H), 4.66 (s, 2H), 4.08-3.97 (m, 4H), 3.80 (s, 3H), 3.24 (t, 2H), 2.83 (d, 2H), 2.58-2.53 (m, 2H), 1.96-1.84 (m, 4H), 1.66 (d, 2H), 1.55-1.47 (m, 1H), 1.30-1.22 (m, 8H)

¹³C-NMR (75 MHz, CDCl₃): δ 165.0, 157.7, 147.6, 133.1, 112.3, 111.6, 98.1, 61.9, 56.4, 53.2, 52.0, 45.3, 36.4, 29.5, 24.9, 23.1, 16.8

MS 462.1 [M+H]⁺

Conversion to the hydrochloride salt was effected using ethereal HCI.

### EXAMPLE 42

### Preparation of intermediate 1-benzyl-4-carbonylamide piperidine

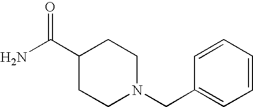

To a stirred suspension of isonipectamide (16.5 g, 0.13 mol) and K₂CO₃ (35.6 g, 0.26 mol) in EtOH (350 ml) was added benzylbromide (22.0 g, 0.13 mol) and the mixture was heated under reflux for 3 h, cooled to room temperature and filtered. The filtrate was evaporated in vacuo and added H₂O (200 ml) was added. The aqueous layer was extracted with CH₂Cl₂ (3×150 ml), the organic layers combined and dried over Na₂SO₄ and filtered. The solvent was evaporated in vacuo to leave the product as a white solid (20.0 g, 71.0%).

¹H-NMR (300 MHz, CDCl₃): δ 7.34-7.21 (m, 5H), 6.36 (br S, 1H), 5.80 (br s, 1H), 3.49 (s, 2H), 2.92 (d, 2H), 2.14-1.99 (m, 1H), 1.96 (t, 2H), 1.85-1.72 (m, 4H)

### EXAMPLE 43

### Preparation of intermediate 1-benzyl-4-cyano-piperidine

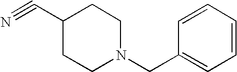

1-Benzyl-4-carbonylamide piperidine (20.0 g, 91.7 mmol) was mixed with P₂O₅ (16.92, 119.2 mmol) and heated under argon at 180-200° C. for 3 h, cooled to room temperature and H₂O (150 ml) was added. The aqueous solution was basified by careful addition of K₂CO₃ and then extracted with EtOAc (3×150 ml). The organic extracts were dried over Na₂SO₄, filtered and the solvent evaporated in vacuo to leave a yellow oil (16.7 g, 90.9%).

¹H-NMR (200 MHz, CDCl₃): δ 7.41-7.25 (m, 5H), 3.53 (s, 2H), 2.75-2.64 (m, 2H), 2.40-2.34 (m, 2H), 1.98-1.86 (m, 5H)

### EXAMPLE 44

### Preparation of intermediate 1-benzyl-4-aminomethylpiperidine

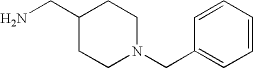

To a suspension of LiAlH₄ (4.84 g, 0.128 mol) in dry Et₂O (40 ml) under an argon atmosphere at 0° C. was dropwise added a solution of 1-benzyl-4-cyano-piperidine (18.3 g, 91.5 mmol) in dry Et₂O (80 ml) and the mixture was stirred at room temperature for 24 h. The reaction mixture was treated carefully with H₂O (10 ml), 10% aqueous NaOH (10 ml) and H₂O (30 ml) to give a mineral precipitate. The precipitate was filtered through a pad of kieselguhr, washed with Et₂O and the filtrate evaporated in vacuo to leave the product as an oil (21.4 g, 82.3%).

¹H-NMR (200 MHz, CDCl₃): δ 7.37-7.22 (m, 5H), 6.42 (br s, 1H), 5.84 (br s, 1H), 3.51 (s, 2H), 2.94 (d, 2H), 2.16-1.67 (m, 7H)

### EXAMPLE 45

### Preparation of intermediate methyl 2-(3-chloropropoxy) indole-3-carboxylate

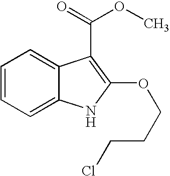

A suspension of methyl indole-3-carboxylate (5.25 g, 30.0 mmol) and DABCO (1.84 g, 16.4 mmol) in dry CH₂Cl₂ (25 ml) was cooled to 0° C. under an argon atmosphere, treated in one portion with NCS (4.41 g, 33.0 mmol) and the mixture stirred for 10 min. The resulting solution was added to a solution of 3-chloropropan-1-ol (3.12 g, 33.0 mmol) in dry CH₂Cl₂ (25 ml) containing anhydrous methane sulphonic acid (0.23 ml). The resulting suspension was stirred for 30 min and then washed with 10% aqueous Na₂CO₃ solution (3×25 ml). The organic layer was dried over Na₂SO₄, filtered and concentrated in vacuo. The resulting oil was triturated with toluene (10 ml) at 0° C. for 1 h and the solid precipitate filtered, washed with a small amount of toluene and dried in vacuo to leave the product as an off-white solid (5.22 g, 65.0%).

¹H-NMR (200 MHz, CDCl₃): δ 9.51 (s, 1H), 8.04 (d, 1H), 7.28-7.14 (m, 3H), 4.49 (t, 2H), 3.96 (s, 3H), 3.67 (t, 2H), 2.18-2.10 (m, 2H) 20

### EXAMPLE 46

### Preparation of intermediate methyl 3,4-dihydro-2H-[1,3]oxazino[3,2-a]indole-10-carboxylate

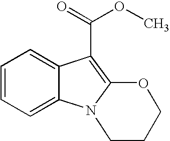

Methyl 2-(3-chloropropoxy)indole-3-carboxylate (5.0 g, 18.7 mmol) was added to a stirred mixture of 5.4 M aqueous NaOH (3.8 ml) and toluene (50 ml) and heated at 40° C. for 4 h. The aqueous layer was separated and the organic layer washed with H₂O (3×25 ml) while maintaining the temperature at 60° C. The organic solvent was evaporated in vacuo to leave the product as a white solid (4.0 g, 93.2%).

¹H-NMR (200 MHz, CDCl₃): δ 8.0 (dd, 1H), 7.24-7.12 (m, 3H), 4.50 (t, 2H), 4.06 (t, 2H), 3.91 (s, 3H), 2.34-2.26 (m, 2H)

### EXAMPLE 47

### Preparation of intermediate 3,4-dihydro-N-[1-(phenylmethoxy)-4-piperidinyl]methyl]-2H-[1,3]oxazino [3,2-a]indole-10-carboxamide

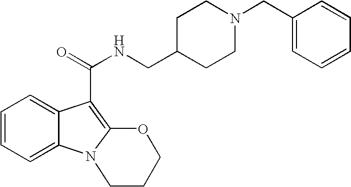

Trimethylaluminium (2 M in toluene, 9 ml) was diluted with dry toluene (9 ml) and the solution cooled to 0° C. under an argon atmosphere. 1-Benzyl-4-aminomethylpiperidine (from Example 46) (3.37 g, 16.5 mmol) was added to the solution, followed by methyl 3,4-dihydro-2H-[1,3]oxazino[3,2-a]indole-10-carboxylate (from Example 48) (3.81 g, 16.5 mmol). The reaction mixture was heated under reflux for 5 h, cooled to room temperature and 10% aqueous NaOH solution (40 ml) was added dropwise. The toluene layer was washed with H₂O, brine and evaporated in vacuo to give an oil. The residue was purified by flash chromatography (SiO₂, CH₂Cl₂/MeOH (7:3)) to leave the product as an off white solid (3.52 g, 53.4%).

¹H-NMR (300 MHz, DMSO-d₆): δ 8.34 (d, 1H), 7.33-7.06 (m, 8H), 6.53 (t, 1H), 4.49 (t, 2H), 4.04 (t, 2H), 3.51 (s, 3H), 3.34 (t, 2H), 2.92 (d, 2H), 2.36-2.28 (q, 2H), 2.03-1.95 (m, 2H), 1.78-1.62 (m, 3H), 1.43-1.34 (m, 2H)

### EXAMPLE 48

### Preparation of intermediate 3,4-dihydro-N-[4-piperidinyl]methyl]-2H-[1,3]oxazino[3,2-a]indole-10-carboxamide

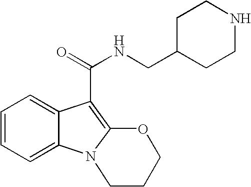

To a stirred solution of 3,4-dihydro-N-[1-(benzyl)-4-piperidinyl]methyl]-2H-[1,3]oxazino [3,2-a]indole-10-carboxamide (2.01 g, 5.0 mol) in EtOH (20 ml) was added hydrazine monohydrate (0.36 ml) and 10% palladium on activated charcoal (M-type, 0.40 g) and the mixture was heated under reflux for 2 h. The reaction mixture was cooled to room temperature, filtered through a pad of kieselguhr and the filtrate evaporated in vacuo to leave the expected product as a white solid (1.52 g, 97.3%).

¹H-NMR (200 MHz, DMSO-d₆): δ 8.09-8.05 (m, 1H), 7.31-7.27 (m, 1H), 7.14-7.03 (m, 2H), 6.81 (t, 1H), 4.59 (t, 1H), 4.23-4.17 (m, 1H), 4.11 (t, 2H), 3.17 (t, 2H), 3.0 (d, 2H), 2.56-2.45 (m, 2H), 2.35-2.24 (m, 2H), 1.66-1.61 (m, 3H), 1.23-1.04 (m, 2H)

### EXAMPLE 49

Alkylation of 3,4-dihydro-N-[4-piperidinyl]methyl]-2H-[1,3]oxazino[3,2-a]indole-10-carboxamide with ethyl 4-bromobutyrate

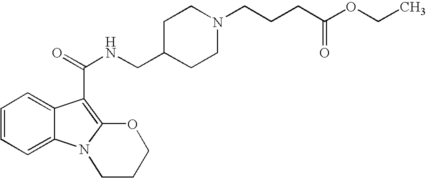

Following the procedure outlined in Example 16, 3,4-dihydro-N-[4-piperidinyl]methyl]-2H-[1,3]oxazino[3,2-a]indole-10-carboxamide (0.62 g, 2.0 mmol) was converted to the title compound as a colourless oil that crystallized upon standing (0.74 g, 86.5%).

¹H-NMR (300 MHz, CDCl₃): δ 8.33 (d, 1H), 7.25-7.10 (m, 3H), 6.56 (t, 1H), 4.55 (t, 2H), 4.14-4.10 (m, 4H), 3.34 (t, 2H), 2.98 (d, 2H), 2.43-2.31 (m, 6H), 2.01 (t, 2H), 1.91-1.81 (m, 4H), 1.73-1.66 (m, 1H), 1.42-1.37 (m, 2H), 1.26 (t, 3H)

¹³C-NMR (75 MHz, CDCl₃): δ 173.4, 164.8, 149.2, 131.0, 125.6, 122.1, 121.0, 120.6, 107.4, 89.2, 66.8, 60.2, 57.8, 53.4, 44.2, 38.9, 36.1, 32.2, 29.7, 22.0, 21.2, 14.2

MS (ES): 450.1 [M+Na]+

Conversion to the HCl-salt was effected with etheral HCl. The precipitate was collected and recrystallized from acetone to leave the HCl-salt as a white crystalline solid.

### EXAMPLE 50

### Alkylation of 3,4-dihydro-N-[4-piperidinyl]methyl]-2H-[1,3]oxazino[3,2-a]indole-10-carboxamide with methyl 6-bromohexanoate

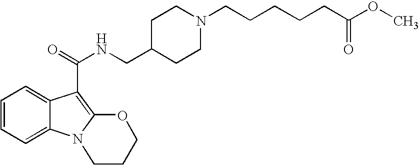

Following the procedure outlined in Example 16, 3,4-dihydro-N-[4-piperidinyl]methyl]-2H-[1,3]oxazino[3,2-a]indole-10-carboxamide (0.31 g, 1.0 mmol) was converted to the title compound as a white solid (0.37 g, 84.5%).

¹H-NMR (300 MHz, CDCl₃): δ 8.33 (d, 1H), 7.23-7.09 (m, 3H), 6.56 (t, 1H), 4.54 (t, 2H), 4.10 (t, 2H), 3.67 (s, 3H), 3.34 (t, 2H), 2.97 (d, 2H), 2.38-2.29 (m, 6H), 1.96 (t, 2H), 1.79 (d, 2H), 1.70-1.30 (m, 10H)

¹³C-NMR (75 MHz, CDCl₃): δ 174.1, 164.7, 149.1, 131.0, 125.5, 122.0, 121.0, 120.6, 107.4, 89.1, 66.8, 58.7, 53.5, 51.4, 44.3, 38.9, 36.2, 33.9, 29.8, 27.1, 26.4, 24.7, 21.2,

### EXAMPLE 51

Alkylation of 3,4-dihydro-N-[4-piperidinyl]methyl]-2H-[1,3]oxazino[3,2-a]indole-10-carboxamide with 2,2,2-trichloroethyl 4-bromobutyrate

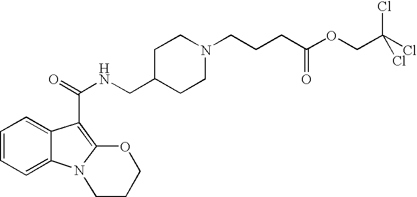

Following the procedure outlined in Example 16, 3,4-dihydro-N-[4-piperidinyl]methyl]-2H-[1,3]oxazino[3,2-a]indole-10-carboxamide (0.31 g, 1.0 mmol) was converted to the title compound as a white solid (0.40 g, 75.8%).

¹H-NMR (300 MHz, CDCl₃): δ 8.32 (d, 1H), 7.27-7.14 (m, 3H), 6.63 (t, 1H), 4.77 (s, 2H), 4.59 (t, 2H), 4.15 (t, 2H), 3.38 (t, 2H), 3.22 (d, 2H), 2.70 (t, 2H), 2.59 (t, 2H), 2.40-2.10 (m, 4H), 2.05-1.96 (m, 2H), 1.91-1.85 (m, 3H), 1.77-1.60 (m, 2H)

¹³C-NMR (75 MHz, CDCl₃): δ 171.7, 166.4, 149.8, 131.5, 125.9, 122.6, 121.2, 121.1, 107.9, 95.2, 89.4, 74.3, 67.3, 57.4, 53.4, 44.2, 39.4, 35.5, 31.8, 28.8, 21.6, 21.1

MS (ES): 553.2 [M+Na]⁺

### EXAMPLE 52

Hydrolysis of the Ethyl Ester from Example 49

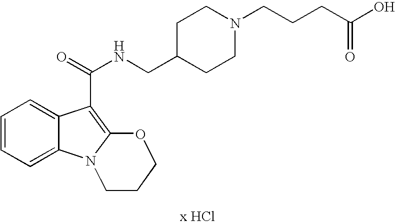

The ethyl ester from Example 49 (0.51 g, 1.20 mmol) was added to a mixture of 2 M aqueous NaOH solution (1.2 ml) and MeOH (5 ml) and refluxed for 2 h. The reaction mixture was cooled to room temperature, concentrated in vacuo and 10% aqueous HCl was added dropwise to pH 2. The precipitate was filtered off, washed with water and dried in vacuo to a white crystalline solid (0.31 g, 72.9%).

¹H-NMR (300 MHz, DMSO-d₆): δ 12.31 (br s, 1H), 10.25 (br s, 1H), 8.10-8.04 (m, 1H), 7.32-7.26 (m, 1H), 7.14-7.04 (m, 2H), 6.96 (t, 1H), 4.59 (t, 2H), 4.15 (t, 2H), 3.43-3-01 (m, 8H), 2.38-2.28 (m, 4H), 1.97-1.81 (m, 5H), 1.68-1.55 (m, 2H)

¹³C-NMR (75 MHz, DMSO-d₆): δ 174.3, 164.6, 150.6, 131.8, 126.1, 122.0, 120.7, 120.4, 109.3, 88.7, 67.9, 56.1, 52.4, 43.7, 34.9, 31.5, 27.7, 21.4, 19.7

MS (ES): 398.1 [M+H]⁺

### EXAMPLE 53

### Alkylation of 3,4-dihydro-N-[4-piperidinyl]methyl]-2H-[1,3]oxazino[3,2-a]indole-10-carboxamide with diethyl 2-bromoethylphosphonate

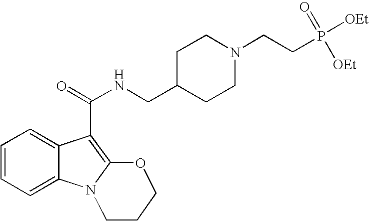

Following the procedure outlined in Example 16, 3,4-dihydro-N-[4-piperidinyl]methyl]-2H-[1,3]oxazino[3,2-a]indole-10-carboxamide (0.29 g, 0.92 mmol) was converted to the title compound as a white solid (0.36 g, 82.9%).

¹H-NMR (300 MHz, CDCl₃): δ 8.30 (d, 1H), 7.23-7.10 (m, 3H), 6.59 (t, 1H), 4.56 (t, 2H), 4.17-4.06 (m, 6H), 3-35 (t, 2H), 3.09 (d, 2H), 2.86-2.78 (q, 2H), 2.39-2.33 (m, 2H), 2.27-2.08 (m, 4H), 1.88-1.60 (m, 3H), 1.58-1-50 (m, 2H), 1.33 (t, 6H)

¹³C-NMR (75 MHz, CDCl₃): δ 163.5, 149.8, 131.5, 125.9, 122.5, 121.3, 121.1, 107.9, 89.4, 67.3, 62.4, 62.3, 53.2, 52.1, 44.3, 39.4, 35.9, 29.3, 21.6, 16.9, 16.8

MS (ES): 500.1 [M+Na]⁺

### EXAMPLE 54

### Preparation of intermediate N-[1-(benzyl)-4-piperidinyl]methyl-1,4-benzodiozane-5-carboxamide

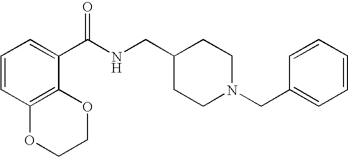

A suspension of 1,4-benzodioxan-5-carboxylic acid (1.80 g, 10.0 mmol) and 1,1-carbonyldiimidazole (1.78 g, 11.0 mmol) in CH₃CN (100 ml) was it was stirred at room temperature for 2 h. 1-Benzyl-4-aminomethylpiperidine (from Example 46) (2.04 g, 10.0 mmol) in CH₃CN (10 ml) was added to the mixture and it was stirred overnight at room temperature. The reaction mixture was concentrated in vacuo, EtOAc (200 ml) was added and the mixture was washed with H₂O (3×50 ml). The organic layer was dried over Na₂SO₄ and evaporated in vacuo to a solid material. The residue was separated with flash chromatography (SiO₂, EtOAc:MeOH, 1:1) to leave the product as a white solid (2.31 g, 63.1%).

¹H-NMR. (200 MHz, CDCl₃): δ 7.74 (dd, 1H), 7.67 (t, 1H), 7.34-7.15 (m, 5H), 7.03-6.89 (m, 2H), 4.43-4.39 (m, 2H), 4.33-4.29 (m, 2H), 3.52 (s, 2H), 3.37 (t, 2H), 2.93 (d, 2H), 2.06-1.93 (m, 2H), 1.77-1.50 (m, 3H), 1.47-1.28 (m, 2H)

### EXAMPLE 55

### Preparation of intermediate N-[4-piperidinyl]methyl]-1,4-benzodioxane-5-carboxamide hydrochloride

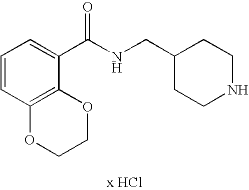

Following the procedure outlined in Example 15, N-[1-(benzyl)-4-piperidinyl]methyl]-1,4-benzodioxane-5-carboxamide (1.88 g, 5.13 mmol) was converted to the title compound as a white solid (1.36 g, 85.2%).

¹H-NMR (300 MHz, CDCl₃): δ 9.68 (br s, 1H), 9.37 (br s, 1H), 7.79 (t, 1H), 7.71-7.68 (dd, 1H), 7.03-6.91 (m, 2H), 4.47-4.45 (m, 2H), 4.34-4.31 (m, 2H), 3.52 (d, 2H), 3.40 (t, 2H), 2.94-2.82 (q, 2H), 2.12-1.69 (m, 5H)

### EXAMPLE 56

### Alkylation of N-[4-piperidinyl]methyl]-1,4-benzodioxane-5-carboxamide hydrochloride with ethyl 4-bromobutyrate

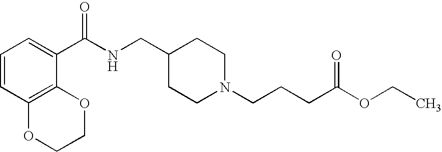

Following the procedure outlined in Example 16, N-[4-piperidinyl]methyl]-1,4-benzodioxane-5-carboxamide hydrochloride (0.56 g, 2.0 mmol) was converted to the title compound as an oil (0.66 g, 85.3%).

¹H-NMR (300 MHz, CDCl₃): δ 7.74-7.71 (dd, 1H), 7.66 (t, 1H), 7.01-6.90 (m, 2H), 4.44-4.41 (m, 2H), 4.33-4.30 (m, 2H), 4.16-4.09 (q, 2H), 3.35 (t, 2H), 2.92 (d, 2H), 2.38-2.30 (m, 4H), 2.02-1.50 (m, 7H), 1.38-1.27 (m, 2H), 1.25 (t, 3H)

¹³C-NMR (75 MHz, CDCl₃): δ 173.5, 164.8, 143.4, 141.8, 124.0, 122.2, 121.3, 120.5, 64.9, 63.5, 60.2, 57.9, 53.3, 45.2, 36.0, 32.3, 29.9, 22.2, 14.1

MS (ES): 413.2 [M+Na]⁺

### EXAMPLE 57

### Alkylation of N-[4-piperidinyl]methyl]-1,4-benzodioxane-5-carboxamide hydrochloride with diethyl 2-bromoethylphosphonate

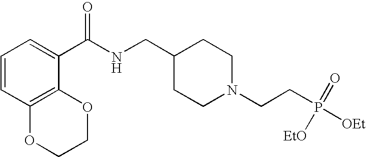

Following the procedure outlined in Example 16, N-[4-piperidinyl]methyl]-1,4-benzodioxane-5-carboxamide hydrochloride (0.71 g, 2.5 mmol) was converted to the title compound as an white solid (0.88 g, 80.7%).

¹H-NMR (300 MHz. CDCl₃): δ 7.74-7.71 (dd, 1H), 7.66 (t, 1H), 6.98-6.93 (m, 2H), 4.43-4.41 (m, 2H), 4.33-4.30 (m, 2H), 4.12-4.05 (m, 6H), 3.35 (t, 2H), 2.92 (d, 2H), 2.58-2.45 (m, 2H), 2.08-1.90 (m, 4H), 1.71-1.50 (m, 3H), 1.36-1.31 (m, 6H)

¹³C-NMR (75 MHz. CDCl₃): δ 165.2, 143.9, 142.3, 135.7, 127.6, 124.5, 122.6, 121.7, 121.0, 65.3, 63.9, 62.0, 61.9, 53.3, 52.1, 45.5, 36.3, 30.3, 16.7

MS (ES): 463.2 [M+Na]⁺

### EXAMPLE 58

### Preparation of intermediate N-[1-(benzyl)-4-piperidinyl]methyl]indole-3-carboxamide

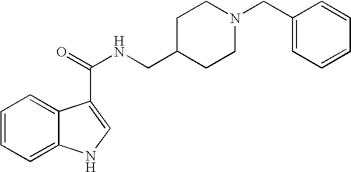

Following the procedure outlined in Example 12, indole-3-carboxylic acid (5.56 g, 31.0 mmol) was converted to the title compound as an oil (3.78 g, 35.0%).

¹H-NMR (300 MHz, CDCl₃): δ 9.96 (s, 1H), 7.96 (d, 1H), 7.67 (s, 1H), 7.44-7.22 (m, 8H), 6.24 (t, 1H), 3.51 (s, 2H), 3.40 (t, 2H), 2.92 (d, 2H), 1.98 (t, 2H), 1.78-1.67 (m, 3H), 1.44-1.30 (m, 2H)

### EXAMPLE 59

### Preparation of intermediate N-[4-piperidinyl]methyl]indole-3-carboxamide

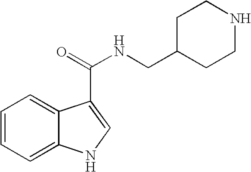

Following the procedure outlined in Example 48, N-[(1-benzyl-4-piperidinyl)methyl]indole-3-carboxamide (1.50 g, 4.3 mol) was converted to the title compound as a white solid (1.07 g, 96.7%).

¹H-NMR (300 MHz, DMSO-d₆): δ 11.56 (br s, 1H), 8.15-8.12 (m, 1H), 8.03 (s, 1H), 7.85 (t, 1H), 7.41 (d, 1H), 7.15-7.08 (m, 2H), 3.12 (t, 2H), 2.92 (d, 2H), 2.55-2.49 (m, 1H), 2.41 (t, 2H), 1.64-1.60 (m, 3H), 1.06-1.01 (m, 2H)

### EXAMPLE 60

Alkylation of N-[4-piperidinyl]methyl]indole-3-carboxamide with ethyl 4-bromobutyrate

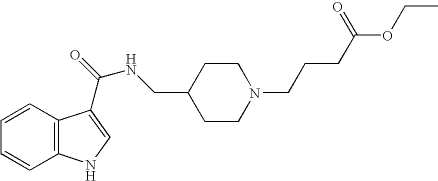

Following the procedure outlined in Example 18, N-[4-piperidinyl]methyl]indole-3-carboxamide (0.24 g, 0.94 mol) was converted to the title compound as a white solid (0.16 g, 47.1%).

¹H-NMR (300 MHz, CDCl₃): δ 9.70 (br s, 1H), 8.00-7.95 (m, 1H), 7.78 (s, 1H), 7.49-7.44 (m, 1H), 7.30-7.25 (m, 2H), 6.33 (t, 1H), 4.20-4.09 (q, 2H), 3.40 (t, 2H), 2.97 (d, 2H), 2.44-2.31 (m, 4H), 1.99-1.76 (m, 7H), 1.43-1.24 (m, 5H)

¹³C-NMR (75 MHz, CDCl₃): δ 173.4, 165.7, 136.4, 128.1, 124.7, 122.7, 121.4, 119.8, 112.1, 60.3, 57.8, 53.2, 44.8, 36.1, 32.2, 29.7, 21.9, 14.2

MS (ES): 394.1 [M+Na]⁺

### EXAMPLE 61

### Alkylation of N-[4-piperidinyl]methyl]indole-3-carboxamide with 2,2,2-trichloroethyl 4-bromobutyrate

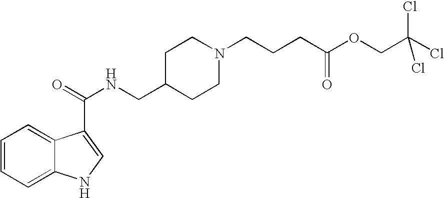

Following the procedure outlined in Example 16, N-[4-piperidinyl]methyl]indole-3-carboxamide (0.94 g, 3.65 mmol) was converted to the title compound as a white solid (0.84 g, 48.4%).

¹H-NMR (300 MHz, CDCl₃): δ 10.08 (br s, 1H), 7.95-7.92 (m, 1H), 7.72 (s, 1H), 7.43 7.40 (m, 1H), 7.23-7.20 (m, 2H), 6.31 (t, 1H), 4.15 (s, 2H), 3-36 (t, 2H), 2.90 (d, 2H), 2.36-2.17 (m, 4H), 1.89 (t, 2H), 1.83-1.65 (m, 5H), 1.36-1.32 (m, 2H)

¹³C-NMR (75 MHz, CDCl₃): δ 173.9, 166.0, 136.5, 128.4, 124.6, 122.6, 121.4, 119.6, 112.2, 111.7, 99.7, 57.8, 53.2, 51.5, 44.9, 36.1, 32.0, 30.8, 29.7, 21.9

MS (ES): 497.2 [M+Na]⁺

### EXAMPLE 62

### Hydrolysis of the Trichloroethyl Ester from Example 61

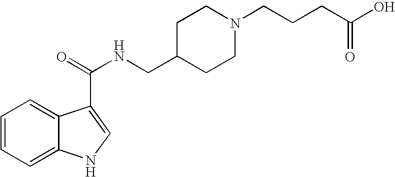

Following the procedure outlined in Example 13, the trichloroethyl ester from Example 61 (0.47 g, 1.0 mmol) was converted to the title compound as a white solid (0.21 g, 61.1%).

¹H-NMR (300 MHz, DMSO-d): δ 11.63 (s, 1H), 8.13 (d, 1H), 8.05 (d, 1H), 7.97 (t, 1H), 7.41 (d, 1H), 7.15-7.05 (m, 2H), 3.14 (t, 2H), 3.02 (d, 2H), 2.50 (t, 2H), 2.26 (t, 2H), 2.17 (t, 2H), 1.75-1.53 (m, 5H), 1.31-1.21 (m, 2H)

¹³C-NMR (75 MHz, DMSO-d₆): δ 174.4, 164.6, 136.0, 127.5, 126.1, 121.6, 120.9, 120.1, 111.7, 110.5, 57.0, 52.2, 43.6, 35.5, 33.4, 28.7, 20.9

MS (ES): 366.2 [M+Na]⁺

### EXAMPLE 63

### Tegaserod

### Preparation of the primary amine 2,2,2-trichloroethyl 5-aminopentanoate

To a stirred solution of 2,2,2-trichloroethyl 5-bromopentanoate (prepared by the same method as in Example 10) in acetone is added potassium phthalimide and the mixture is stirred overnight. The reaction mixture is filtered and the solvent evaporated in vacuo. The residue is added to EtOAc and washed with H₂O. The organic layer is dried over Na₂SO₄ filtered and evaporated in vacuo to leave the title compound. Standard hydrazinolysis in EtOH gives the primary amine as follows.

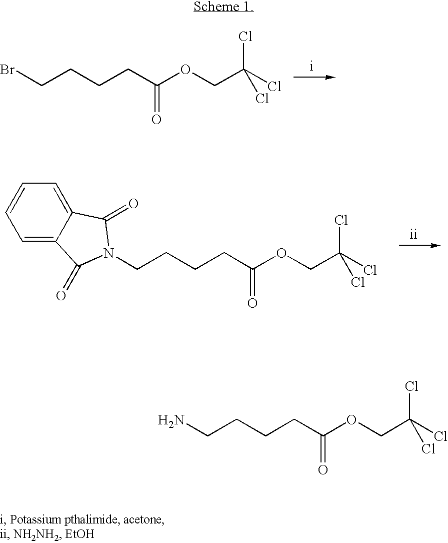

### EXAMPLE 64

### Tegaserod

### Preparation of the monoalkylated amine N-2,2,2-(trichloroethyl pentanoate)-N′-10 aminoguanidine

To a suspension of thiosemicarbamide is added MeI in EtOH and the mixture is heated at 60° C. for ½ h and cooled to room temperature. The resulting suspension is filtered and the filtrate washed with Et₂O to leave S-methyl isothiosemicarbazide hydroiodide. S-methyl isothiosemicarbazide hydroiodide is used in the next step without any further purification. To a solution of this compound in MeOH is added 2,2,2-trichloroethyl 5-aminopentanoate (from Example 63) and the mixture is heated under reflux overnight. The reaction mixture is cooled to room temperature and the solvent evaporated in vacuo to leave the title compound. The amine is used in the next step without any further purification as follows.

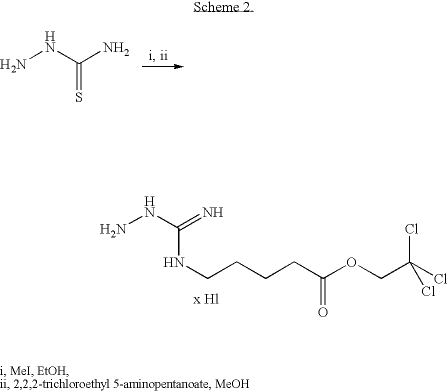

### EXAMPLE 65

### Tegaserod

### Synthesis of the Tegaserod Derivative

To a stirred solution of 5-methoxyindole-3-carboxaldehyde in MeOH is added N-2,2,2-(trichloroethyl pentanoate)-N-aminoguanidine at room temperature. The solution is acidified with conc, aqueous HCl and is stirred overnight. The solvent is evaporated in vacuo and MeOH is added. To the solution is added etheral HCl and the precipitate is filtered off. The precipitate is recrystallized from MeOH/Et₂O to leave the HCl salt of the trichloroethyl ester. This compound is added to a suspension of Zn and a mixture of 1 M aqueous KH₂PO₄ and THF and stirred overnight. The suspension is filtered through a pad of kieselguhr and the solvent evaporated in vacuo. The residue is separated with flash chromatography to leave the title compound as a free acid.

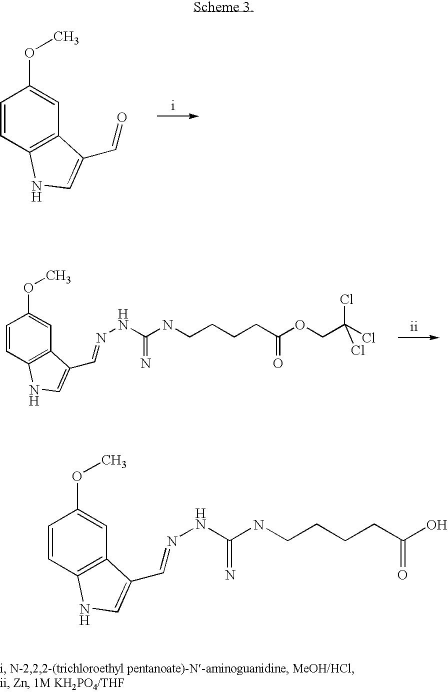

### EXAMPLE 66

### Preparation of a Piperazine Intermediate

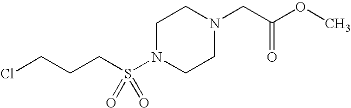

Methyl bromoacetate (6.11 g, 40.0 mmol) was added to a solution of piperazine (34.45 g, 0.40 mol) in THF (250 ml) and heated under reflux for 4 h, cooled to room temperature and evaporated in vacuo. The residue was separated with flash chromatography (SiO₂, CH₂Cl₂/MeOH, 9:1) to give the monoalkylated intermediate. 3-Chloropropanesulfonyl chloride (0.81 g, 4.59 mmol) was dropwise added to a solution of the monoalkylated piperazine derivative (0.66 g, 4.17 mmol) in CH₂Cl₂ (20 ml) at 0° C. The reaction mixture was stirred to room temperature for 3 h, poured into H₂O and the organic layer separated. The CH₂Cl₂ extract was washed with brine, dried over MgSO₄ and evaporated in vacuo to leave the title compound as an oil (1.13 g, 92.6%).

MS (ES) 299 [M+H]⁺

### EXAMPLE 67

### Synthesis of a Benzoate Derivative

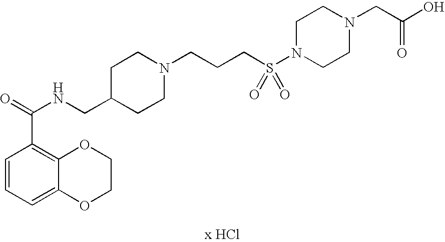

Following the procedure outlined in example 16, N-[4-piperidinyl]methyl]-1,4-benzodioxane-5-carboxamide hydrochloride was alkylated with the piperazine intermediate from example 66. Subsequent alkali hydrolysis, following the procedure in example 52, gave the compound as a white solid (0.75 g, 67.5%)

¹H-NMR (300 MHz, DMSO-_{d6}):

δ 11.13 (br s, 1H), 8.21 (t, 2H), 7.17 (m, 1H), 6.96-6.82 (m, 2H), 4.34-4.26 (m, 4H), 3.61 (br s, 2H), 3.45-3.26 (m, 8H), 3.15-3.00 (m, 6H), 2.98-2.90 (m, 4H), 2.20-2.12 (m, 2H), 1.93-1.55 (m, 5H)

¹³C-NMR (75 MHz, DMSO-d₆):

δ 170.3, 165.7, 144.3, 142.3, 125.3, 122.5, 121.3, 119.8, 65.3, 64.5, 57.5, 55.0, 52.2, 52.1, 49.4, 46.8, 44.6, 34.4, 27.4, 18.5

### EXAMPLE 68

Alkylation of 3-(1,2,3,6-tetrahydropyridin-4-yl)-1H-indole with methyl (4-bromomethyl)benzoate

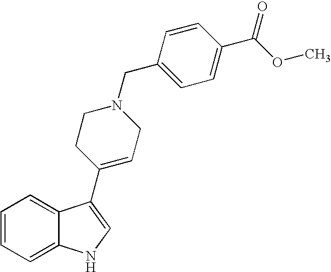

Following the procedure outlined in example 16, 3-(1,2,3,6-tetrahydropyridin-4-yl)-1H-indole was converted to the methyl ester as a yellow solid (1.25 g, 72.3%).

¹H-NMR (300 MHz, DMSO-d₆):

δ 11.10 (br s, 1H), 7.94 (d, 2H), 7.79 (d, 1H), 7.51 (d, 2H), 7.36 (d, 2H), 7.12-6.99 (m, 2H), 6.11 (t, 1H), 3.85 (s, 3H), 3.66 (s, 2H), 3.12 (br s, 2H), 2.66 (t, 2H), 2.53-2.49 (m, 2H)

¹³C-NMR (75 MHz, DMSO-d₆):

δ 166.1, 144.5, 136.9, 129.6, 129.1, 128.8, 128.2, 124.6, 122.7, 121.1, 120.0, 119.1, 117.4, 115.8, 111.6, 61.4, 52.7, 51.9, 49.8, 28.5

### EXAMPLE 69

### Hydrolysis of the Methyl Ester from Example 68

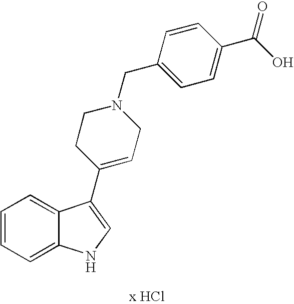

Following the procedure outlined in example 52, the methyl ester from example 68 was converted to the free acid as a yellow solid (0.37 g, 69.4%)

¹H-NMR (300 MHz, DMSO-_{d6}):

δ 11.33 (br s, 1H), 7.95 (d, 2H), 7.78 (d, 1H), 7.65 (d, 2H), 7.39 (d, 2H), 7.12-6.99 (m, 2H), 6.10 (t, 1H), 3.99 (br s, 2H), 3.37 (br s, 2H), 3.16 (s, 3H), 2.88 (br s, 2H), 2.67 (br s, 2H)

¹³C-NMR (75 MHz, DMSO-_{d6}):

δ 167.2, 136.9, 130.7, 129.7, 129.3, 124.4, 123.2, 121.2, 119.9, 119.3, 115.0, 111.8, 59.7, 51.2, 49.1, 48.5, 26.7

MS (ES): 333.0 [M+H]⁺

### EXAMPLE 70

### Alkylation of 3-(1,2,3,6-tetrahydropyridin-4-yl)-1H-indole with ethyl 4-bromobutyrate

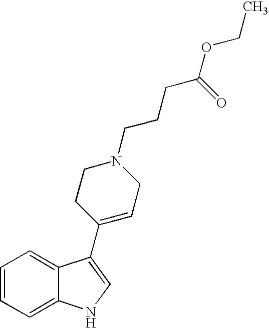

Following the procedure outlined in example 16, 3-(1,2,3,6-tetrahydropyridin-4-yl)-1H-indole was converted to the ethyl ester as a yellow solid (1.03 g, 74.3%).

¹H-NMR (300 MHz, DMSO-_{d6}):

δ 11.18 (br s, 1H), 7.83 (d, 1H), 7.41 (d, 2H), 7.15-7.02 (m, 2H), 6.13 (t, 1H), 3.59 (s, 3H), 3.26 (br s, 2H), 2.79 (br s, 2H), 2.56 (br s, 4H), 2.34 (t, 2H), 1.56-1.54 (m, 4H)

¹³C-NMR (75 MHz, DMSO-d₆):

δ 173.2, 145.1, 136.9, 129.6, 124.5, 122.9, 121.2, 119.9, 119.2, 116.0, 115.4, 111.7, 56.7, 52.0, 51.1, 49.6, 32.9, 27.6, 25.1, 22.2

MS (ES): 313.0 [M+H]⁺

### EXAMPLE 71

### Preparation of an Adamantane Carboxylic Acid Intermediate

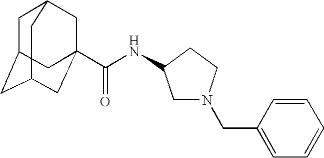

1,1′-Carbonyldiimidazole (0.65 g, 3.92 mmol) was added to a solution of 1-adamantanecarboxylic acid (0.59 g, 3.27 mmol) in DMF (20 ml) and stirred at room temperature for 1 h. R-(−)-1-Benzyl-3-aminopyrrolidine (0.67 g, 3.60 mmol) was added and the resulting mixture stirred overnight, evaporated in vacuo and the residue separated with flash chromatography (SiO₂, CH₂Cl₂:MeOH, 9:1) to leave the product as a white solid (0.87 g, 79.2%)

¹H-NMR (300 MHz, DMSO-d₆):

δ 7.33-7.23 (m, 5H), 4.17-4.10 (m, 1H), 3.54 (s, 2H), 2.68 (t, 2H), 2.55-2.45 (m, 3H), 2.22-2.17 (m, 1H), 2.03-1.94 (m, 4H), 1.78-1.60 (m, 15H)

### EXAMPLE 72

### Synthesis of an Adamantane Carboxylic Acid Derivative

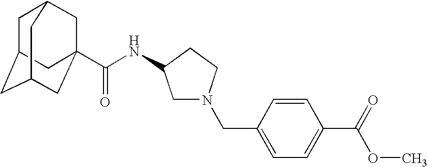

Following the procedure outlined in example 48, the benzyl intermediate from example 71 was converted to the free amine. This was alkylated with methyl (4-bromomethyl)benzoate to give the product as a yellow oil (0.64 g, 80.5%)

¹H-NMR (200 MHz, CDCl₃):

δ 7.95 (d, 2H), 7.36 (d, 2H), 5.94 (d, 1H), 4.41 (br s, 1H), 3.87 (s, 3H), 3.63 (s, 2H), 2.84 (t, 1H), 2.55-2.52 (m, 2H), 2.29-2.21 (m, 2H), 2.04-1.99 (m, 3H), 1.78-1.55 (m, 12H)

### EXAMPLE 73

### Preparation of a Tetrahydrobenzindole Intermediate

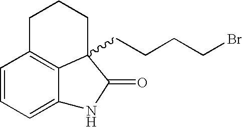

NaH powder (252 mg, 5.73 mmol) was added to a solution of 2A,3,4,5-tetrahydrobenz(CD)indol-2(1H)-one (1.0 g, 5.73 mmol) in dry DMF (10 ml) under argon atmosphere at 0° C. and stirred for 1 h. 1,4-Dibromobutane (3.4 ml, 28.65 mmol) was added and the reaction mixture stirred for additional 1 h, poured into H₂O and extracted with EtOAc. The combined organic layers were washed with brine, dried over MgSO₄ and evaporated in vacuo. The residue was separated with flash chromatography (SiO₂, Hexane:EtOAc, 4:1) to leave the intermediate as a white solid (0.59 g, 33.4%)

¹H-NMR (300 MHz, CDCl₃):

δ 8.04 (br s, 1H), 7.16 (t, 1H), 6.82 (d, 1H), 6.71 (d, 1H), 3.31 (t, 2H), 2.83-2.75 (m, 1H), 2.70-2.62 (m, 1H), 2.14-2.02 (m, 2H), 1.89-1.77 (m, 5H), 1.45-1.15 (m, 3H)

### EXAMPLE 74

### Preparation of a Tetrahydrobenzindole Intermediate

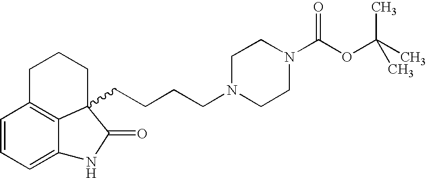

Following the procedure outlined in example 16, the intermediate from example 73 was alkylated with 1-boc-piperazine to give the piperazine intermediate as a colorless oil (0.42 g, 53.2%).

¹H-NMR (300 MHz, CDCl₃):

δ7.77 (br s, 1H), 7.14 (t, 1H), 6.82 (d, 1H), 6.70 (d, 1H), 3.49-3.39 (m, 4H), 2.92-2.55 (m, 2H), 2.40-2.27 (m, 5H), 2.15-2.08 (m, 3H), 1.91-1.82 (m, 3H), 1.50-1.24 (m, 14H)

### EXAMPLE 75

### Synthesis of a Tetrahydrobenzindole Derivative

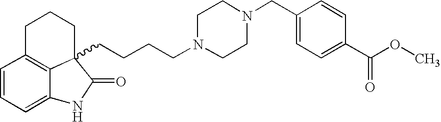

Following the procedure outlined in example 39, the boc-group was cleaved with 4 M HCl in dioxane to give the free piperazine amine as a white crystalline solid. This amine was alkylated with methyl (4-bromomethyl)benzoate to give the methyl ester as a white solid (0.19 g, 40.8%)

¹H-NMR (200 MHz, CDCl₃):

δ 7.95 (d, 2H), 7.70 (br s, 1H), 7.34 (d, 2H), 7.08 (t, 1H), 6.76 (d, 1H), 6.64 (d, 1H), 3.88 (s, 3H), 3.52 (s, 2H), 2.93-2.54 (m, 10H), 2.40-2.32 (m, 2H), 2.07-1.95 (m, 2H), 1.84-1.73 (m, 3H), 1.48-1.08 (m, 5H)

### EXAMPLE 76

### Hydrolysis of the Methyl Ester from Example 75

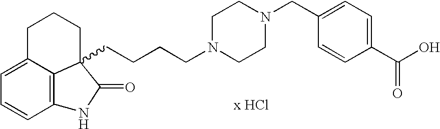

Following the procedure outlined in example 52, the methyl ester from example 75 was converted to the free acid as a white solid (60 mg, 22.1%).

¹H-NMR (200 MHz, DMSO-d₆):

δ 10.14 (s, 1H), 7.87 (d, 2H), 7.35 (d, 2H), 7.03 (t, 1H), 6.69 (d, 1H), 6.59 (d, 1H), 3.50 (br s, 2H), 3.14 (s, 4H), 2.79-2.68 (m, 1H), 2.55-2.20 (m, 7H), 2.10-1.62 (m, 5H), 1.35-0.88 (m, 6H)

### EXAMPLE 77

### Preparation of intermediate 4-(4-bromophenyl)-1-benzyl-1,2,5,6-tetrahydropyridine

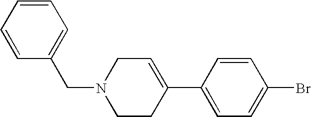

Benzylbromide (8.88 g, 51.94 mmol) was added to a stirred suspension of 4-(4-bromophenyl)-4-hydroxypiperidine (11.09 g, 43.29 mmol) and K₂CO₃ (8.97 g, 64.93 mmol) in DMF (100 ml) at 0° C. The reaction mixture was stirred to room temperature overnight, diluted with H₂0 (100 ml) and extracted with EtOAc (2×75 ml). The combined organic layers were washed with brine, dried over MgSO₄ and evaporated in vacuo to leave 4-(4-bromophenyl)-1-benzyl-4-hydroxypiperidine as a colorless solid (11.43 g, 76.2%). A mixture of 4-(4-bromophenyl)-1-benzyl-4-hydroxypiperidine (11.43 g, 33.0 mmol) and p-toluene-sulfonic acid monohydrate (12.55 g, 66.0 mmol) in toluene (75 ml) was refluxed under Dean-Stark conditions for 1 h. The resulting mixture was cooled to room temperature, diluted with CH₂Cl₂ and washed with brine and H₂O. The organic extract was dried over MgSO₄ and evaporated in vacuo to leave the title compound as a colorless solid.

¹H-NMR (200 MHz, CDCl₃):

δ 7.49-7.27 (m, 9H), 6.13-6.09 (m, 1H), 3.69 (s, 2H), 3.23-3.19 (m, 2H), 2.76 (t, 2H), 2.60-2.50 (m, 2H)

### EXAMPLE 78

### Preparation of intermediate ethyl 4-(1-benzyl-1,2,5,6-tetrahydropyridin-4-yl)benzoate

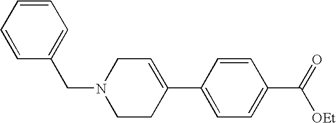

A solution of 4-(4-bromophenyl)-1-benzyl-1,2,5,6-tetrahydropyridine (6.04 g, 18.41 mmol) in dry THF (130 ml) under argon atmosphere was cooled to −78° C. and added n-BuLi (14.95 ml, 23.92 mmol). The reaction mixture was stirred for 15 min, then added diethyl carbonate (23.6 ml, 0.18 mol) and stirred to room temperature for 3 h. The resulting mixture was poured into ice-water and extracted with EtOAc. The organic extract was washed with brine, dried over MgSO₄ and evaporated in vacuo. The residue was separated with flash chromatography (SiO₂, Hexane:EtOAc, 9:1) to leave the title compound as a yellow oil (0.70 g, 11.8%)

¹H-NMR (200 MHz, CDCl₃):

δ 8.02 (d, 2H), 7.50-7.30 (m, 7H), 6.25-6.21 (m, 1H), 4.41 (q, 2H), 3.71 (s, 2H), 3.28-3.23 (m, 2H), 2.79 (t, 2H), 2.66-2.59 (m, 2H), 1.42 (t, 3H)

### EXAMPLE 79

### Preparation of intermediate ethyl 4-(1,2,5,6-tetrahydropyridin-4-yl)benzoate hydrochloride

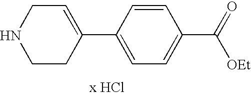

Following the procedure outlined in example 15, ethyl 4-(1-benzyl-1,2,5,6-tetrahydropyridin-4-yl)benzoate is cleaved with α-chloroethyl chloroformate to give the amine as a hydrochloride salt.

### EXAMPLE 80

### Preparation of intermediate 2-tert-butoxycarbonyl-2,3,4,9-tetrahydro-1H-pyrido[3,4-b]indole

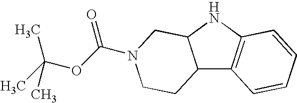

A mixture of 1,2,3,4-tetrahydro-9H-pyrido-[3,4-b]indole, di-tert-butyl dicarbonate and K₂CO₃ in 2-propanol/H₂O is stirred at room temperature overnight. The resulting mixture is poured into EtOAc and then washed with H₂O, dried over MgSO₄ and evaporated in vacuo to leave the title compound as a solid.

### EXAMPLE 81

### Preparation of intermediate 2-tert-butoxycarbonyl-9-methoxycarbonylmethyl-1,2,3,4-tetrahydropyrido[3,4-b]indole

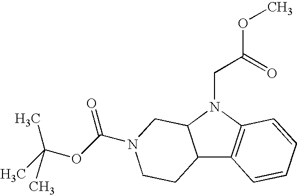

A mixture of 2-tert-butoxycarbonyl-2,3,4,9-tetrahydro-1H-pyrido[3,4-b]indole and NaH powder in dry DMF under argon atmosphere is stirred at room temperature for 30 min and then dropwise added methyl bromoacetate. The resulting mixture is stirred at room temperature overnight and then poured into H₂O. The whole is extracted with EtOAc and the combined organic layers washed with brine, dried over MgSO₄ and evaporated in vacuo. The residue is separated with flash chromatography to leave title compound as a solid.

### EXAMPLE 82

### Preparation of intermediate 9-methoxycarbonylmethyl-1,2,3,4-tetrahydropyrido[3,4-b]indole hydrochloride

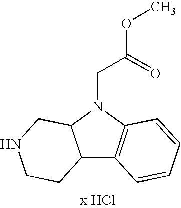

Following the procedure outlined in example 39, the boc-protected amine from example 81 is cleaved with 4 M HCl in dioxane to leave the title compound as a solid.

Ethyl 4-(1,2,5,6-tetrahydropyridin-4-yl)benzoate hydrochloride (from example 79) and 9-methoxycarbonylmethyl-1,2,3,4-tetrahydropyrido[3,4-b]indole hydrochloride (from example 82) are examples of amino compounds (hereafter called amino fragments), that can be coupled with different aromatic fragments to give a wide variety of 5-HT-ligands. The synthesis of the amino fragments prepared in example 79 and 82 are summarized in scheme 6 and 7:

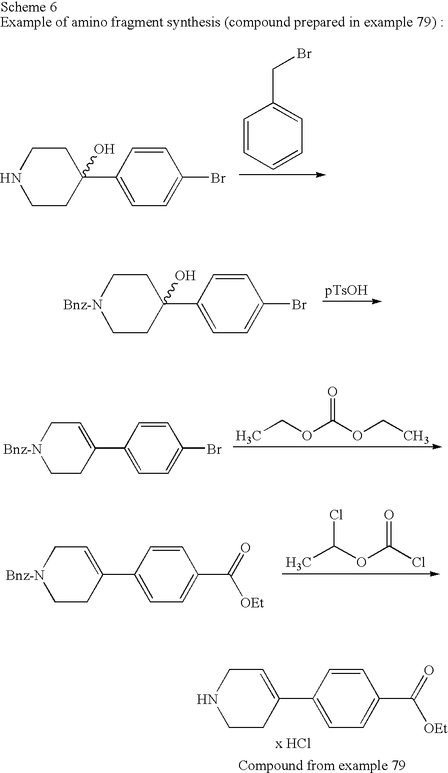

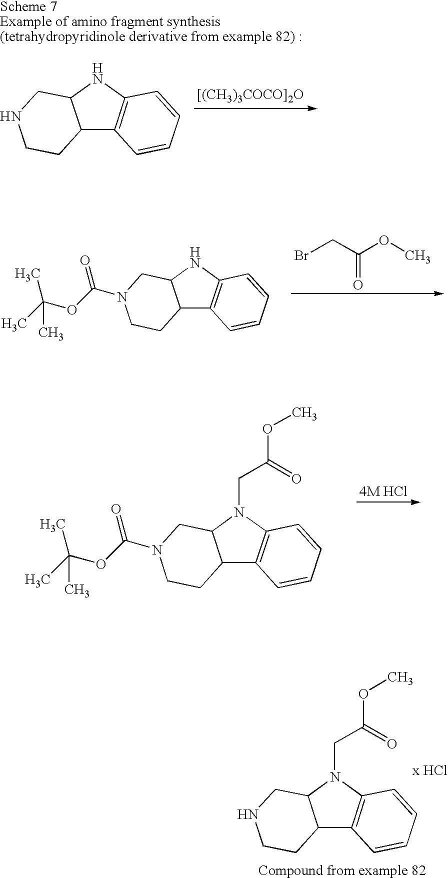

Examples of new hydrophilic tetrahydrobenzindole derivatives and their synthesis are summarized in scheme 8.

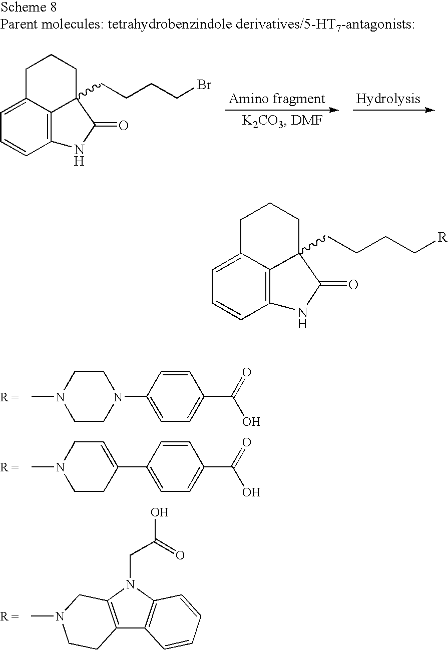

Similar are examples of new hydrophilic long chain arylpiperazine derivatives (LCAPS) and their synthesis given in scheme 9 (scheme 5 also describes this type of hydrophilic derivatives):

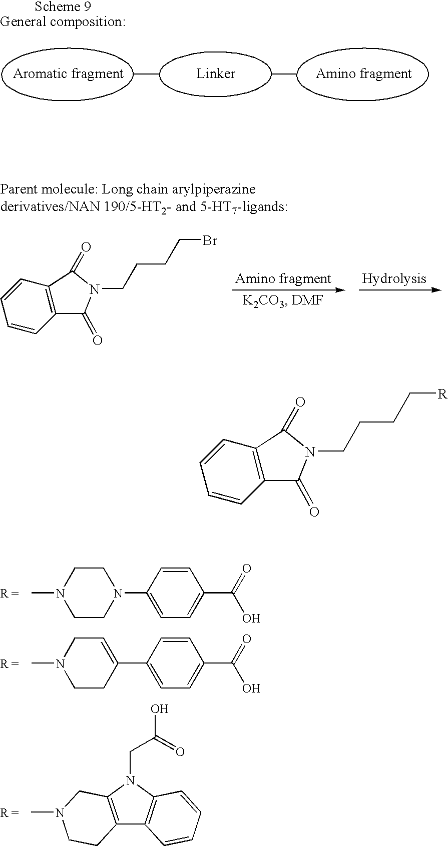

The amino fragments in scheme 6 and 7 can also be used in the synthesis of 5-HT_{2A}-antagonists (ketanserin analogues):

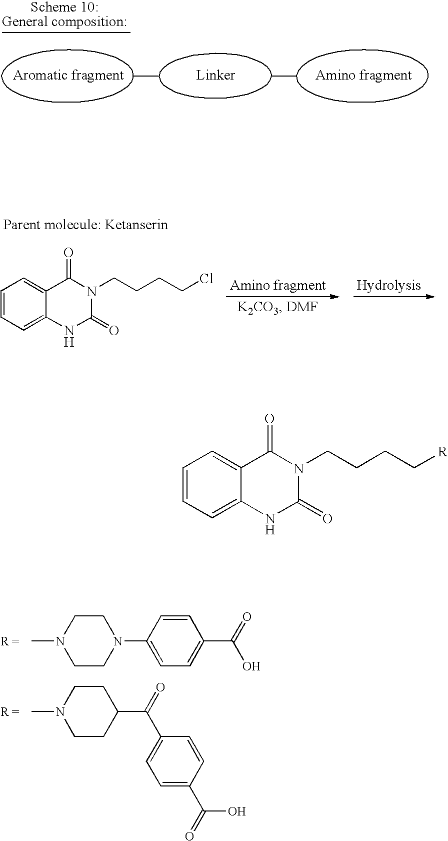

The quinazolinedione group in ketanserin can be replaced with other aromatic fragments:

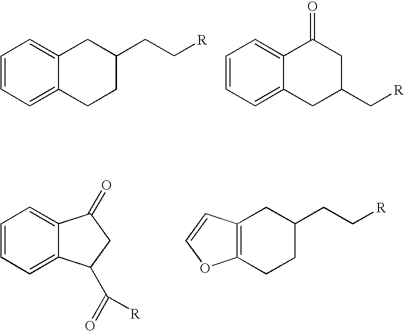

The indole derivative described in scheme 4 (a partial 5-HT₇-agonist) will gain affinity for the 5-HT_{2A}-receptor by derivatization at the indole nitrogen. This can for example be achieved by copper-catalyzed Ullman arylation:

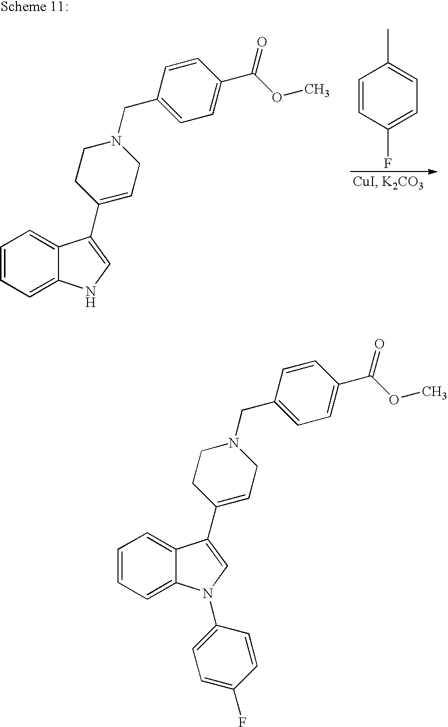

### EXAMPLE 83

In Vitro Biological Testing of Hydrophilic 5-HT₄ Ligands in Binding Assays and Adenylyl Cyclase Assays

### Materials and Methods

### Establishment of HEK293 Cell Lines Stably Expressing Human 5-HT₄ (b) Receptors

The development of HEK293 cell lines stably expressing human 5-HT_{4(b)} receptors was described and published previously (Bach et al. 2001). Briefly, HEK293 cells (ATCC) were grown in Dulbecco's modified Eagle's medium with 10% fetal calf serum and penicillin (100 U/ml) and streptomycin (100 μg/ml). Cells were transfected with plasmid DNA (pcDNA3.1(−) containing human 5-HT_{4(b)} receptor cDNA) using SuperFect Transfection Reagent (QIAGEN) according to the manufacturers protocol. Serial dilutions of transfected cells were plated in 96 well plates containing G418 (geneticin; Amersham) at 0.4 mg/ml, and isolated single colonies of cells transformed to the neomycin-resistant phenotype were expanded and tested for expression of serotonin receptors by measuring serotonin-stimulated adenylyl cyclase activity (Themmen et al. 1993). Transformed cells were always grown in the presence of G418 (0.4 mg/ml). For binding and adenylyl cyclase analysis, stable cell lines were grown and maintained in UltraCULTURE™ general purpose serum-free medium (BioWhittaker, Walkersville, Md., USA), supplemented with L-glutamine (2 mM), penicillin (100 U/ml) and streptomycin (100 μg/ml).

### Membrane Preparation for Radioligand Binding and Adenylyl Cyclase Assay

Membranes were prepared from stably transfected HEK293 cells cultured on 150-mm cell culture dishes and grown to 80% confluence in serum-free medium (UltraCULTURE™, BioWhittaker) with penicillin (10 U/ml) and 2 mM L-Glutamine (BioWhittaker). Cells were washed twice with 10 ml ice-cold HBSS, scraped with a rubber policeman in 10 ml ice-cold HBSS and collected by centrifugation at 800 g for 5 min at 4° C. The cell pellet was resuspended in 1 ml/dish ice-cold STE buffer (27% (w/v) sucrose, 50 mM Tris-HCl, pH 7.5 at 20° C., 5 mM EDTA) and homogenized with an Ultra-Turrax (IKA) homogenizer, using five 10 s bursts with 30 s cooling in ice-water between bursts. To remove nuclei, the homogenate was centrifuged at 300 g for 5 min at 4° C. and the supernatant was further centrifuged at 17000 g for 20 min at 4° C. and the supernatant removed. The crude membrane pellet was resuspended with ten strokes of tight fitting pestle B in a Dounce glass-glass homogenizer in 1 ml/dish ice-cold TE (50 mM Tris-HCl, pH 7.5 at RT, 5 mM EDTA). This procedure was repeated twice and the resuspended membranes were finally aliqouted and flash frozen in liquid nitrogen and stored at −70° C. until use.

### Radioligand Binding Assay

Binding assays were performed in 96-well, round-bottom microtiter plates with total reaction volumes of 50-200 μl, containing the indicated concentration of [³H]GR113808 with or without competing unlabelled ligand in a binding buffer containing 50 mM Tris-HCl (pH 7.5 at RT), 1 mM EDTA, 5 mM EGTA, 2 mM MgCl₂, 1 mM ascorbate, 0.1% BSA and 100 μM GTP. The plates were incubated at 23° C. for 60 min and harvested onto UniFilter™-96 GF/C™ (Packard Instrument Co., Meriden, Conn., USA), presoaked in 0.3% polyethyleneimine (Sigma), using a Packard FilterMate Universal Harvester with 96-well format, and washed 4-6 times with approximately 0.25 ml/well of ice-cold buffer, containing 50 mM Tris-HCl (pH 7.0 at RT) and 2 mM MgCl₂. The filters were dried and counted at approximately 40% efficiency in a Top-Count liquid scintillation counter (Packard), using 20 μl per filter well of Micro-Scint liquid scintillation cocktail (Packard).

### Adenylyl Cyclase Assay

Adenylyl cyclase activity was measured by determining conversion of [α-³²P]ATP to [³²P]cAMP in membranes prepared in STE by homogenization of cells grown and washed as described above in a Dounce glass-glass homogenizer by 10 strokes with the tight-fitting pestle. Membranes were kept on ice prior to assay. Adenylyl cyclase activities were measured on 10-μl aliquots in a final volume of 50 μl in the presence of 0.1 mM [α-³²P]ATP (1-2×10⁶ cpm/assay), 4 mM MgCl₂, 20 μM GTP, 1 mM EDTA, 1 mM [³H]cAMP (ca. 10,000 cpm/assay), 1 μM 3-isobutyl-1-methyl xanthine (IBMX; Sigma), a nucleoside triphosphate regenerating system consisting of 20 mM creatine phosphate (Sigma), 0.2 mg/ml creatine phosphokinase (Sigma) and 40 U/ml myokinase (Sigma) and additives described in the text and figures. When forskolin (Calbiochem, La Jolla, Calif., USA) was used the concentration was 100 μM. Incubations were for 20 min at 32° C. Cyclic AMP formed was quantified by the double column chromatography system of Salomon et al. (1974) as modified by Bockaert et al. (1976).

### Analysis of Binding and Adenylyl Cyclase Data

Binding and adenylyl cyclase data were analyzed by non-linear regression using Microsoft Excel with the Solver add-in, using the below equations.

Competitive binding assays—The data were fit to the equation

Y=a+(b−a)/(1+x/c) [1]

where a is non-specific binding, b is total binding in the absence of competitor, c is IC₅₀, and x is the concentration of competitor. Where relevant, relative binding data were obtained by recalculating the data using a=0 and b=100.

Activation of adenylyl cyclase—The data were fit to the equation

Y=a+(b−a)x/(c+x) [2]

where a is basal adenylyl cyclase activity, b is maximal adenylyl cyclase activity stimulated by the agonist, c is EC₅₀, and x is the concentration of agonist.

IC₅₀ values from competitive binding assays were converted to Kb values by the method of Cheng and Prusoff (1973).

### Protein Measurements

The protein concentrations in the membrane preparations were measured with the Micro BCA Protein Assay Reagent Kit (Pierce, Rockford, Ill., USA) using bovine serum albumin (BSA) as standard.

Radiochemicals

[³H]GR113808 (84 Ci/mmol), [α-³²P]ATP (400 Ci/mmol) and [³H]cAMP (30-50 Ci/mmol) were from Amersham (Buckinghamshire, England).

### Compounds

5-Hydroxytryptamine hydrochloride (5-HT, serotonin) was from Sigma (St. Louis, Mo., USA). GR113808 (1-methyl-1H-indole-3-carboxylic acid, [1-[2-[(methylsulfonyl)amino]ethyl]-4-piperidinyl]methyl ester) maleate was from Tocris (Avonmouth, UK). The other compounds tested were synthesized by Drug Discovery Laboratories AS (DDL) (Oslo, Norway).

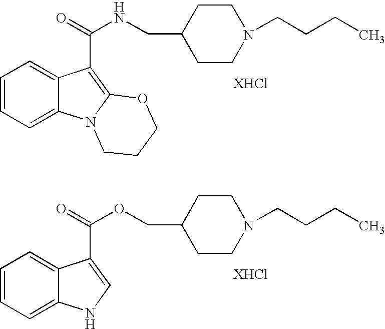

### Standards

DDL-6001 (piboserod) DDL-6002

Results of In Vitro Biological Testing of 5-HT₄ Ligands In Adenylyl Cyclase and Binding Assays, Organised Per Compound (Table 1)

TABLE 1

| Substance | Antagonist | | Agonist/ | Binding affinity | |
|---|---|---|---|---|---|
| from | pK_{b} value | | Antagonist | (pK_{d} value) | |
| Example | (pK_{b} ± SEM¹) | n | properties | (pK_{d} ± SEM¹) | n |
| GR113808 | 9.27 ± 0.06 | 12 | Antagonist | 10.09 ± 0.07 | 5 |
| SB207266 | 9.26 ± 0.08 | 13 | Antagonist | 10.15 ± 0.15 | 6 |
| (piboserod) | | | | | |
| 12 | 9.13 ± 0.04 | 2 | Antagonist | 8.30 ± 0.12 | 3 |
| 13 | 8.15 ± 0.08 | 2 | Antagonist | 9.04 ± 0.16 | 3 |
| 60 | 5.79 ± 0.21 | 2 | Antagonist | 6.96 ± 0.05 | 3 |
| 62 | 4.55 ± 0.26 | 2 | Unknown | 5.44 ± 0.01 | 3 |
| 18 | 5.49 | 1 | Antagonist | 5.80 ± 0.18 | 2 |
| 17 | 4.42 | 1 | | 6.00 | 1 |
| 22 | 7.95 | 1 | Weak partial | 7.37 ± 0.01 | 2 |
| | | | agonist | | |
| 26 | 7.16 ± 0.04 | 2 | Weak partial | 7.89 ± 0.19 | 2 |
| | | | agonist | | |
| 25 | 8.18 | 1 | Antagonist | 8.19 ± 0.11 | 2 |
| 27 | 7.72 | 1 | Weak partial | 7.80 ± 0.24 | 2 |
| | | | agonist | | |
| 29 | 6.51 | 1 | Partial agonist | 6.70 ± 0.30 | 2 |
| 31 | 6.75 | 1 | Partial agonist | 7.17 ± 0.25 | 2 |
| 33 | 5.98 | 1 | Partial agonist | 6.16 ± 0.08 | 2 |
| 35 | 6.14 | 1 | Partial agonist | 5.92 ± 0.03 | 2 |
| 36 | 5.94 | 1 | Partial agonist | 5.66 ± 0.14 | 2 |
| 40 | 7.18 | 1 | Partial agonist | 7.70 ± 0.09 | 2 |
| 47 | 9.37 ± 0.15 | 3 | Antagonist | 9.80 ± 0.19 | 4 |
| 49 | 8.81 ± 0.38 | 3 | Antagonist | 9.91 ± 0.17 | 4 |
| 51 | 9.27 ± 0.21 | 2 | Antagonist | 9.58 ± 0.16 | 4 |
| 50 | 9.12 ± 0.12 | 2 | Antagonist | 9.87 ± 0.16 | 4 |
| 53 | 8.60 ± 0.18 | 2 | Antagonist | 8.88 ± 0.10 | 3 |
| 52 | 7.96 ± 0.02 | 2 | Antagonist | 8.68 ± 0.08 | 4 |
| 67 | 8.56 ± 0.32 | 2 | Antagonist | 9.09 ± 0.13 | 3 |

¹In the experiments where n = 2, the values are given as pK_{b}/pK_{d} ± half-range

### EXAMPLE 84

### In Vitro Biological Testing of Hydrophilic 5-HT₁ and 5-HT_{2A} Ligands in Binding Assays

### Materials and Methods

The same materials and methods described in example 83 were used for the biological testing of hydrophilic 5-HT₇ and 5-HT_{2A} ligands. Membrane preparations of HEK293 cell lines stably expressing human 5-HT₇₍ₐ₎ receptors (Krobert et al. 2001), or HEK293 cells transiently transfected with human 5-HT_{2A} receptor were used in the binding assays. 5-Carboxamido [³H]tryptamine trifluoroacetate ([³H]5-CT) and [³H]ketanserin were used as radioligands in binding assays testing 5-HT₇ and 5-HT_{2A} ligands, respectively.

### Radiochemicals

[³H]5-CT (91 Ci/mmol) was from Amersham (Buckinghamshire, England). [³H]ketanserin (72.2 Ci/mmol) was from Perkin Elmer (Boston, Mass., USA).

### Compounds

5-Hydroxytryptamine hydrochloride (5-HT, serotonin) was from Sigma (St. Louis, Mo., USA). RS102221 (8-(5-(2,4-Dimethoxy-5-(4-trifluoromethylphenylsulphonamido)phenyl-5-oxopentyl]-1,3,8-triazaspiro[4.5]decane-2,4-dione hydrochloride) was from Tocris (Avonmouth, UK). The other compounds tested were synthesized by Drug Discovery Laboratories AS (DDL) (Oslo, Norway).

### Results of In Vitro Biological Testing of New 5-HT₂/5-HT₇ Ligands in Binding Assays, Organised Per Compound (Table 2)

TABLE 2

| | |
|---|---|
| Substance from | Binding affinity (pK_{d} value) |

| | | |
|---|---|---|
| example | (pK_{d} ± SEM¹) | N |

5-HT_{2A} ligands

| | | |
|---|---|---|
| RS102221 | 5.34 ± 0.02 | 2 |
| 72 | 4.31 ± 0.03 | 2 |

5-HT₇ ligands

| | | |
|---|---|---|
| 5-HT | 8.55 ± 0.16 | 2 |
| 9 | 5.07 ± 0.09 | 2 |
| 68 | 4.94 ± 0.05 | 2 |
| 69 | 4.09 ± 0.18 | 2 |
| 70 | 6.18 ± 0.12 | 2 |
| 75 | 4.80 ± 0.06 | 2 |
| 76 | 3.87 ± 0.01 | 2 |

¹In the experiments the values are given as pK_{d} ± half-range

1. An oxyacid or oxyacid ester 5-HT receptor modulator or a physiologically tolerable salt thereof.

2. A modulator according to claim 1 wherein said modulator is not a 5-HT₄ receptor modulator.

3. The modulator according to claim 1 wherein said modulator is a 5-HT₂ receptor modulator.

4. The modulator according to claim 1 wherein said modulator is a 5-HT₇ receptor modulator.

5. The modulator according to claim 1 wherein said modulator is a 5-HT₁ receptor modulator.

6. The modulator according to claim 1, wherein the oxyacid group is attached via a linker group to the parent 5-HT receptor molecule and wherein said linker is selected from the group consisting of straight chain or branched optionally substituted C₁₋₁₀ alkyl, C₂₋₁₀ alkenyl and C₂₋₁₀ alkynyl, optionally attached via an amino, oxy, carbonyl, oxycarbonyl, carbonyloxy, aminocarbonyl or carbonylamino, group.

7. The modulator according to claim 6 wherein the oxyacid group is spaced away from a pharmacophore of the parent 5-HT receptor modulator molecule by at least three consecutive bonds.

8. The modulator according to claim 6 wherein the oxyacid group is spaced away from a pharmacophore of the parent 5-HT receptor modulator molecule by at least five consecutive bonds.

9. The modulator according to claim 1, wherein the parent receptor modulator comprises an indole ring or a 2-oxa equivalent and wherein the oxyacid group is attached via the 3-position, or if at the 1-position by a group providing at least 6 bonds spacing from the indole ring nitrogen.

10. The modulator according to claim 1, wherein the parent receptor modulator comprises a 4-phenyl-piperazin-1-yl group wherein the oxyacid group is attached via the 1-position nitrogen.

11. The modulator according to claim 1, wherein the parent receptor modulator comprises an indolinyl or quinazolinyl group wherein the oxyacid is attached via the 3-position.

12. The modulator according to claim 1, wherein the parent receptor modulator comprises a benzo- or dibenzo-azepinyl group wherein the oxyacid is attached via the 2-position.

13. The modulator according to claim 1, wherein the parent receptor modulator comprises a phthalimide group wherein the oxyacid is attached via the phthalimide nitrogen.

14. The modulator according to claim 1, wherein the parent receptor modulator comprises a 1,2,3,4-tetrahydronaphthalene group, or 4-oxo equivalent, wherein the oxyacid is attached via the 2-position.

15. The modulator according to claim 1, wherein the parent receptor modulator comprises an indane group, or 3-oxo equivalent, wherein the oxyacid is attached via the 1-position.

16. The modulator according to claim 1, wherein the parent receptor modulator comprises a 4, 5, 6, 7-tetrahydrobenzofuran group wherein the oxyacid is attached via the 5-position.

17. The modulator according to claim 1, wherein the oxyacid group has a pKₐ of no more than 6.4.

18. The modulator according to claim 1 which is selected from the group consisting of 4N-[3-(2-aminoethyl)-1H-indole-5-carboxamide]butanoic acid, 3-(1-carboxy-1,2,3,6-tetrahydropyridin-4-yl)-5-methoxy-1H-indole, 1-carboxy-4-[N-[2-[4-(2-methoxyphenyl)-1-piperazinyl]ethyl]-N-2-pyridinyl]cyclohexanecarboxamide, 3-[2-(dicarboxymethylamino)ethyl]-N-methyl-1H-indole-5-methanesulfonamide, 4N-[3-(2-aminopropyl)-1H-indol-5-ol]butanoic acid, N-[(4-carboxyphenyl)methyl]-5-methoxy-1H-indole-3-ethanamine, 3-[2-[4-(4-carboxybenzoyl)-1-piperidinyl]ethyl-2,4(1H, 3H)-quinazolinedione, 6-(4-(4-carboxybutyl)-1-piperazinyl)-11H-dibenz[b,e]azepine and 3-(1-(4-carboxybenxyl)-1,2,3,6-tetrahydropyridin-4-yl)-5-methoxy-1H-indole.

19. A process for the production of a hydrophilic analogue of a 5-HT receptor modulator as defined in claim 1, said process comprising
(a) reacting said receptor modulator with a bifunctional reagent comprising a modulator binding functional group and an optionally protected oxyacid group; or
(b) reacting an intermediate in the preparation of said receptor modulator with a bifunctional reagent comprising an intermediate binding functional group and an optionally protected oxyacid group, and optionally further reacting the resultant compound to produce said analogue; and, optionally,
(c) removing or replacing the oxyacid protecting groups.

20. A pharmaceutical composition comprising a receptor modulator or salt thereof according to claim 1 together with at least one physiologically tolerable carrier or excipient.

21. (canceled)

22. (canceled)

23. The method of claim 28, wherein the modulator is a 5-HT_{1B} or _{1D} agonist which is administered outside the CNS.

24. The method of claim 28, wherein the modulator is a 5-HT_{1B} or _{1D} antagonist.

25. (canceled)

26. (canceled)

27. (canceled)

28. A method of preventing a human or non-human mammalian subject from contracting a serotonin-related condition which method comprises administering on one side of the blood brain barrier an effective amount of a receptor modulator or salt thereof according to claim 1.

29. The method of claim 28, wherein said modulator or salt is a 5-HT₂ or 5-HT₇ receptor modulator which is administered outside the CNS.

30. A method as claimed in claim 28 wherein said modulator or salt is a 5-HT₄ receptor modulator which is administered on the CNS side of the blood brain barrier.
